(19) United States
(12) Patent Application Publication
(10) Pub. No.: US 2022/0177837 A1
(43) Pub. Date: Jun. 9, 2022

(54) GEOMETRIC INDUCTION OF PLURIPOTENCY

(71) Applicants: UNIVERSITA' DEGLI STUDI DI MILANO, Milano (IT); POLITECNICO DI MILANO, Milano (IT); CONSIGLIO NAZIONALE DELLE RICERCHE, Roma (IT)

(72) Inventors: Stefania CARELLI, Milano (IT); Manuela Teresa RAIMONDI, Milano (IT); Anna Maria DI GIULIO, Milano (IT); Toniella GIALLONGO, Milano (IT); Alfredo GORIO, Milano (IT); Giulio Nicola CERULLO, Milano (IT); Roberto OSELLAME, Roma (IT)

(21) Appl. No.: 17/437,004

(22) PCT Filed: Mar. 9, 2020
(86) PCT No.: PCT/IB2020/052021

§ 371 Date: Sep. 7, 2021

(30) Foreign Application Priority Data
Mar. 8, 2019 (IT) 102019000003377

(51) Int. Cl. C12N 5/0797; C12N 5/0775

(52) U.S. Cl. CPC C12N 5/0623; C12N 5/0667; C12N 2506/1384; C12N 2501/115; C12N 2501/11

(57) ABSTRACT

A method for culturing cells on a substrate capable of inducing pluripotency is provided. The method includes plating cells on a nichoid-type substrate, allowing cultured cells to proliferate for a certain period of time, detaching cells from the nichoid-type substrate, and, once cells have been detached, culturing cells in suspension or under adhesion.

### BACKGROUND ART

Neurodegenerative diseases represent a severe threat for human health. Parkinson's disease (PD) is the second most common neurodegenerative disease after Alzheimer's disease. The main pathological finding is the degeneration of the dopaminergic neurons of Substantia Nigra pars compacta (SNpc) which leads to the loss of dopamine in the striatum. Several drugs are available for managing motor and non-motor symptoms of Parkinson's disease. However, all are aimed at alleviating symptoms in improving the patients' quality of life. At this time, no disease-modifying treatment or therapy is available. Cell therapies have been considered a feasible regenerative approach to compensate for the loss of SNpc dopaminergic neurons in PD. The existence of a subclass of neural progenitors derived from the subventricular zone (derived from SVZ) surviving after donor death has been successfully reported (Marfia G et al. Adult neural precursors isolated from post mortem brain yield mostly neurons: an erythropoietin-dependent process. Neurobiol Dis. 2011; 43(1):86-98). These post-mortem neural precursors which physiologically release erythropoietin (Er-NPCs) show a high neuronal differentiation, which depends on the release of autocrine erythropoietin (EPO), since it is blocked when the cells themselves are exposed to anti-EPO or anti-EPO-R antibodies. The therapeutic potential of Er-NPCs was demonstrated in a pre-clinical experimental model of PD, in which cells were unilaterally transplanted into the striatum of C57/black mice exposed to MPTP. Er-NPCs-treated animals had a quick behavioral improvement within the third day after cell transplantation (Carelli S et al. Grafted Neural Precursors Integrate Into Mouse Striatum, Differentiate and Promote Recovery of Function Through Release of Erythropoietin in MPTP-Treated Mice. ASN Neuro. 2016 Oct. 27; 8(5); Recovery from experimental parkinsonism by intrastriatal application of erythropoietin or EPO-releasing neural precursors. Neuropharmacology. 2017 June; 119:76-90). The same cells were also tested with positive results in the pre-clinical model of traumatic spinal cord injury (Carelli S et al. Exogenous adult postmortem neural precursors attenuate secondary degeneration and promote myelin sparing and functional recovery following experimental spinal cord injury. Cell Transplant. 2015; 24(4): 703-19. Carelli et al. EPO-releasing neural precursor cells promote axonal regeneration and recovery of function in spinal cord traumatic injury. Restor Neurol Neurosci. 2017; 35(6):583-599). Recently, there have been technological innovations, which allow neural stem cells to be cultured in three dimensions, to produce organoids which represent various human tissues, even the brain. These substrates for the generation of three-dimensional organoids, which recapitulate the brain allow to shape and study the cell-cell interactions and complex cyto-architecture more in detail and in more physiological contexts, as they are of the same size as the cell, compared to traditional tissue culture systems. Three-dimensional microstructuring of the material by two-photon polymerization induced by femtosecond laser (2PP) is emerging as an important tool in biomedicine. As a rapid prototyping technique, two-photon polymerization allows the fabrication of three-dimensional microstructures and nanostructures directly from computer-generated models, with a spatial resolution of up to 100 nm (Raimondi M T et al. Two-photon laser polymerization: from fundamentals to biomedical application in tissue engineering and regenerative medicine. J Appl Biomater Funct Mater. 2012 Jun. 26; 10(1):55-65).

The technique was successfully applied to the production of three-dimensional microscaffolds, or “synthetic niches”, using an organic-inorganic hybrid polymer material referred to as SZ2080. This scaffold fabricated by the 2PP technique, referred to as a nichoid, has shown a good ability to promote the spontaneous formation of stem colonies, promote cell proliferation, and preserve the staminality of rat primary mesenchymal stem cells, mesenchymal cells derived from human bone marrow, and mouse embryonic stem cells (Raimondi M T et al. Three-dimensional structural niches engineered via two-photon laser polymerization promote stem cell homing. Acta Biomater. 2013; 9(1):4579-84; Raimondi M T et al. Optimization of direct laser-written structural niches to control mesenchymal stromal cell fate in culture. Micromachines, 2014, Vol. 5; Nava M M et al. Synthetic niche substrates engineered via two-photon laser polymerization for the expansion of human mesenchymal stromal cells. J Tissue Eng Regen Med. 2017; 11(10):2836-2845; Nava M M et al. Two-photon polymerized “nichoid” substrates maintain function of pluripotent stem cells when expanded under feeder-free conditions. Stem Cell Res Ther. 2016; 7(1):132; Nava M M et al. Interactions between structural and chemical biomimetism in synthetic stem cell niches. Biomed Mater. 2015; 10(1):015012).

Recently, the same results were also obtained with murine and mesenchymal neural stem cells isolated from human adipose tissue (data not yet published).

WO 2017/037108 describes nichoids and their use for the cultivation of stem cells, especially both adult stem cells, more particularly mesenchymal and neural stem cells, and embryonic stem cells. The trials described included the expansion of said cells on said nichoids for the whole duration of the trial itself and the maintenance of the differentiation.

The need to have methods and devices allowing the control of the fate of stem cells in culture (proliferation, staminality maintenance (pluripotency and multipotency), in particular of adult stem cells, in an effective and reproducible manner, is strongly felt. Such a control would favor both the biological research and the efficacy of cell therapies, in which the stem cells are the therapeutic agent.

In particular, there is a strong need for methodologies for the reprogramming of cells, which can thus be differentiated towards the condition of pluripotency, and for differentiating them from the condition of pluripotency.

### SUMMARY OF THE INVENTION

The authors of the present invention have surprisingly found that adult stem cells cultured on a nichoid do not only remain more viable than the control, where the same cells were cultured in neurospheres, but in the same cells the nichoid is capable of inducing pluripotency. The authors of the present invention have also surprisingly noted that the pluripotency induction is caused by the geometry of the system, and that there is no exogenous induction of chemical and/or genetic type on the cells.

The authors of the present invention have further found that adult stem cells, proliferated on a nichoid and then detached, surprisingly give rise to a population of viable cells which, once transplanted in vivo, remain viable and do not originate tumors and have a greater therapeutic power than the same cells expanded under standard floating conditions.

### BRIEF DESCRIPTION OF THE DRAWINGS

FIG. 1: Efficiency of the detachment of neural precursors from the nichoid after 7 days in culture. Cell dissociation is given as a percentage with respect to mechanical dissociation. Every condition was tested in triplicate in each trial. The plotted data are the mean of three different trials ±SD. The statistical count significance with respect to mechanical dissociation is expressed by * p<0.05, ** p<0.01, *** p<0.001.

FIG. 2: Viability of neural precursors detached from the nichoid with different methods after 7 days in culture. Cell viability is given as a percentage with respect to mechanical dissociation. Every condition was tested in triplicate in each trial. Data are expressed as mean±SD. * p<0.05, ** p<0.01 and *** p<0.001 with respect to mechanical dissociation.

FIG. 3: Assay of spheroid formation by neural precursors after detachment from the nichoid with different methods. The images of the different dissociation methods are acquired at 2 days. The perfect non-dissociation of most methods quickly leads to the formation of very large spheres and forces the cells to be divided before normal time (7 days). The best choices for dissociation, which are alternative to mechanical detachment, are 10 mM EDTA and citric saline solution. 400 μm bars.

FIG. 4: Percentage of neural precursors detached from the nichoid after 7 days of growth in the nichoid itself with sodium citrate solution and 10 mM EDTA. The percentage of detached cells in the control was 100% for sodium citrate solution and 10 mM EDTA. All cells treated with sodium citrate solution detached from the nichoid, while only 60% of cells treated with 10 mM EDTA do. Every condition was tested in triplicate in each trial. The plotted data are the mean of three different trials ±SD. The statistical count significance with respect to CSS control is expressed by * p<0.05, ** p<0.01, *** p<0.001.

FIG. 5: Viability of neural precursors detached after 7 days with sodium citrate solution and 10 mM EDTA. The percentage of living cells was always about 90%, except in the nichoid treated with 10 mM EDTA, where the viability was only 60-65%. Every condition was tested in triplicate in each trial. Data are expressed as mean±SD. The statistical count significance with respect to CSS control is expressed by * p<0.05, ** p<0.01, *** p<0.001.

FIG. 6: Assay of spheroid formation by neural precursors after detachment from the nichoid with 10 mM EDTA or sodium citrate solution. The images of the two different dissociation methods are acquired at time zero, at 2 and 5 days. The imperfect dissociation of most methods quickly leads to the formation of very large spheres and forces the cells to be divided before normal time (7 days). The best dissociation methods, which are alternative to mechanical dissociation, are 10 mM EDTA and sodium citrate solution. 400 μm bars

FIG. 7: Cell growth in the nichoid for different times. The neural precursors were plated in the medium for neural stem cells at a density of 1×10⁴ cells/cm². The plot shows that the cells plated on the nichoid grow more than the control for all the days analyzed: 3, 7, 10 and 14 days in culture. The analysis was performed three times for every condition. Data are expressed as mean±SD. The statistical significance of the count performed with respect to CSS control is expressed by * p<0.05, ** p<0.01 and *** p<0.001.

FIG. 8: Viability of the detached neural precursors after 3, 7, 10 and 14 days in culture on a nichoid. The number of cells living in the nichoid was always significantly greater at all observation times considered, compared to the control. Every condition was tested in triplicate in each trial. The statistical count significance with respect to CSS control is expressed by * p<0.05, ** p<0.01, *** p<0.001.

FIG. 9: A) Er-NPCs within the nichoid, after 3 days from the plating, start forming neurospheres. 40× magnification. Scale: 100 μm. B): Er-NPCs, after 7 days, form a pad of cells. 20× magnification. Scale: 200 μm.

FIG. 10: After 7 days, Er-NPCs grown within the nichoid exhibited a cell density of about 4×10⁵ cells/cm². Data are expressed as a mean of three independent trials ±SD.

FIG. 11: Assay of spheroid formation with cells kept under floating control conditions and cells grown in the nichoid. After a single day of spheroid formation assay, under both conditions, Er-NPCs form neurospheres again, as shown in the figure. 10× magnification. Scale: 400 μm.

FIG. 12: Assay of spheroid formation with cells kept under floating control conditions and cells grown in the nichoid. On day 3, the spheres formed by Er-NPCs increase, as shown in the figure. 10× magnification. Scale: 400 μm.

FIG. 13: Assay of spheroid formation with cells kept under floating control conditions and cells grown in the nichoid. The cells from the nichoid formed, after 7 days, smaller neurospheres but in greater numbers, instead under control conditions they were larger and with much less viability. 10× magnification. Scale: 400 μm.

FIG. 14: Viability of Er-NPCs grown in the nichoid for 7 days, proliferated outside the nichoid for two weeks. Data are expressed as a mean of three independent trials with similar results ±SD. * p<0.05, ** p<0.01, *** p<0.001 vs control under standard floating condition.

FIG. 15: Erythropoietin (EPO) expression by Western blotting in neural precursors after 7 days of culture on the nichoid compared to control conditions in floating cultures.

FIG. 16: EPO-R expression by Western blotting in neural precursors after 7 days of culture on the nichoid compared to control conditions in floating cultures.

FIG. 17: Immunofluorescence study of the expression of EPO, NESTIN and beta-TUBIII (TUJ1) markers.

FIG. 18: Distribution of the markers investigated in the neural precursors with respect to Z axis.

FIG. 19: Expression of the staminality marker Sox2, quantified by Real time RT-PCR, in neural precursors cultured in the nichoid with respect to the control under fluctuating conditions.

FIG. 20: Expression of the staminality marker Oct4, quantified by Real time RT-PCR, in neural precursors cultured in the nichoid with respect to the control under fluctuating conditions.

FIG. 21: Expression of the staminality marker Nanog, quantified by Real time RT-PCR, in neural precursors cultured in the nichoid with respect to the control under fluctuating conditions.

FIG. 22: Expression of the staminality marker Nestin, quantified by Real time RT-PCR, in neural precursors cultured in the nichoid with respect to the control under fluctuating conditions.

FIG. 23: Expression of Sox2, Nanog, Oct4, Nestin and Tuj1 in neural precursors in the nichoid with respect to control conditions under fluctuating conditions. The evaluations are carried out by Western blotting.

FIG. 24: Number of EPO-positive neural precursors after seven days of culture in the nichoid under differentiated stimuli with a conditioning medium, but without using the biological substrate Matrigel™ (required for the differentiation under standard conditions). The quantification was carried out by the image analysis software ImageJ and shows the percentage of cells which are positive for the markers studied. The quantification indicates that about 80% of differentiated cells are EPO-positive in the control, 82% in the nichoid despite the absence of Matrigel™. Data are expressed as a mean of three independent trials ±SD. * p<0.05, ** p<0.01, *** p<0.001 vs nichoid without Matrigel™.

FIG. 25: Number of BETA TUBIII (TUJ1)-positive neural precursors after seven days of culture in the nichoid under differentiated stimuli with a conditioning medium, but without using the biological substrate Matrigel™. The quantification was carried out by the image analysis software ImageJ and shows the percentage of cells which are positive for the markers studied. The quantification indicates that less than 50% of differentiated cells are TUJ-1-positive in the control, 95% in the nichoid despite the absence of Matrigel™. Data are expressed as a mean of three independent trials ±SD. * p<0.05, ** p<0.01, *** p<0.001 vs nichoid without Matrigel™.

FIG. 26: Number of MAP2-positive neural precursors after seven days of culture in the nichoid under differentiated stimuli with a conditioning medium, but without using the biological substrate Matrigel™. The quantification was carried out by the image analysis software ImageJ and shows the percentage of cells which are positive for the markers studied. The quantification indicates that about 70% of differentiated cells are MAP2-positive in the control, 100% in the nichoid despite the absence of Matrigel™. Data are expressed as a mean of three independent trials ±SD. * p<0.05, ** p<0.01, *** p<0.001 vs nichoid without Matrigel™.

FIG. 27: Count of neural precursors and viability thereof on nichoid and re-plated under floating conditions for 7 days for a new expansion. Data are expressed as a mean of three independent trials ±SD. * p<0.05, ** p<0.01, *** p<0.001 vs control under standard floating conditions.

FIG. 28: Count of neural precursors and viability thereof on nichoid and re-plated under floating conditions for 14 days for a new expansion. Data are expressed as a mean of three independent trials ±SD. * p<0.05, ** p<0.01, *** p<0.001 vs control under standard floating conditions.

FIG. 29: Neurosphere size by neural precursors 7 days after maintenance under fluctuating conditions, post-cultivation in the nichoid for 7 days. Data are expressed as a mean of three independent trials.

FIG. 30: Assay of neurosphere formation by neural precursors 7 days after maintenance under fluctuating conditions, post-cultivation in the nichoid for 7 days. The comparison is carried out with neural precursors always maintained under standard floating conditions.

FIG. 31: Immunofluorescence (EPO, Nestin, Tuj and GFAP) characterization of neurospheres formed by neural precursors maintained in culture for 7 days under floating conditions, post-cultivation in the nichoid for the previous 7 days.

FIG. 32: Expression of staminality factors (Sox2, Oct4, Nanog) by Real-time PCR, in neural precursors maintained in culture for 7 days under floating conditions, post-cultivation in the nichoid for the previous 7 days. Data are expressed as a mean of two independent trials ±SD. * p<0.05, ** p<0.01, *** p<0.001 vs (standard floating) control conditions.

FIG. 33: Therapeutic effect of neural precursors grown in the nichoid and transplanted in an experimental animal model of Parkinson's disease. The cells cultured in the nichoid for 7 days promote the therapeutic effect in mice (in term of function recovery) despite a 72% lower dosage of the administered cells. Number of transplanted animals: 3. * p<0.05, ** p<0.01, *** p<0.001 vs healthy control. ^{∘} p<0.05, ^{∘∘} p<0.01, ^{∘∘∘} p<0.001 vs MPTP (parkinsonian animal).

FIG. 34: Viability of human mesenchymal stem cells derived from adipose tissue and detached from the nichoid with different methods after 7 days of culture. Every condition was tested in triplicate in each trial. Data are expressed as mean±SD. * p<0.05, ** p<0.01 and *** p<0.001 with respect to plastic control.

FIG. 35: Viability and proliferation curves of human mesenchymal stem cells derived from adipose tissue and grown in the nichoid and under standard adherent control conditions up to 14 days in culture. Every condition was tested in triplicate in each trial (number of trials: 3). Data are expressed as mean±SD. * p<0.05, ** p<0.01 and *** p<0.001 with respect to plastic control.

FIG. 36: Expression of GFAP and Vimentin in mesenchymal stem cells derived from human adipose tissue and grown in the nichoid and under standard adherent control conditions up to 7 days of culture. In the plot, the same indicators along Z axis for the nichoid. 120× magnification. Scale bar: 20 μm.

FIG. 37: Expression of beta-actin and GFAP in mesenchymal stem cells derived from human adipose tissue and grown in the nichoid and under standard adherent control conditions up to 7 days of culture. In the plot, the same indicators along Z axis for the nichoid. 120× magnification. Scale bar: 20 μm

FIG. 38: Expression of Sox2 and Nestin in human mesenchymal stem cells derived from adipose tissue and grown in the nichoid and under standard adherent control conditions up to seven days in culture. In the plot, the same indicators along Z axis for the nichoid. 120× magnification. Scale bar: 20 μm

FIG. 39: Expression of Oct4 and Nestin in human mesenchymal stem cells derived from adipose tissue and grown in the nichoid and under standard adherent control conditions up to 7 days of culture. In the plot, the same indicators along Z axis for the nichoid. 120× magnification. Scale bar: 20 μm.

FIG. 40: Immunofluorescence coexpression analysis of NANOG and NESTIN in hADSCs grown for 7 days under standard conditions and within the nichoid. In the plot, the same indicators along Z axis for the nichoid. 120× magnification. Scale bar: 20 μm.

FIG. 41: Viability of mesenchymal stem cells derived from human adipose tissue and maintained in culture for 7 days under adhesion to plastic, after cultivation in the nichoid. Data are expressed as a mean of three independent trials ±SD. * p<0.05, ** p<0.01, *** p<0.001 vs nichoid without Matrigel™.

FIG. 42: hADSCs grown on the nichoid with a different initial concentration. 4× magnification. Scale: 1,000 μm.

FIG. 43: Comparison of hADSCs grown on the nichoid and under standard conditions for 7 days, re-plated under conditions of adhesion to plastic. 4× magnification. 4× magnification. Scale: 1,000 μm.

FIG. 44: Results in the analysis of RNA sequencing.

FIG. 45: mRNA expression of Sox2, Oct4 Nanog in mesenchymal stem cells grown in the nichoid with respect to the control. The results are expressed as a mean±SD of three independent trials carried out in duplicate (n=6; *p<0.05; **p<0.01 vs Control).

FIG. 46: Expression of dopaminergic markers. Histograms relate to the quantification of immunofluorescences performed on striatum sections reacted with anti-tyrosine hydroxylase (TH) and anti-dopamine neurotransmitter transporter antibody. (DAT). a) Quantification of the expression of the marker TH. The histogram shows the percentage of cells, which are positive for the marker TH under different treatment conditions. The results are expressed as mean±SD (n=3: **p<0.01 vs Control, #p<0.05 vs MPTP). The adjacent histogram shows the percentage of positive cells compared to the dose of transplanted NPCs. The results are expressed as mean±SD (n=3; *p<0.05 vs MPTP-NPCs). b) Quantification of the expression of the marker DAT. The histogram shows the percentage of cells, which are positive for the marker DAT under different treatment conditions. The results are expressed as mean±SD (n=3: *p<0.05 vs Control, #p<0.05 vs MPTP). The adjacent histogram shows the percentage of positive cells compared to the dose of transplanted NPCs. The results are expressed as mean±SD (n=3; *p<0.05 vs MPTP-NPCs).

FIG. 47: Quantification of fluorescence intensity related to neuroinflammation markers. The plots show the quantification of the fluorescence performed on striatum sections reacted with anti-GFAP (above, a) and anti-MOMA (below, b) antibodies. The results are expressed as mean±SD (n=3 *p<0.05 vs Control, #p<0.05 vs MPTP).

### OBJECT OF THE INVENTION

A first object of the present invention is a method for inducing pluripotency in stem cells by using a nichoid-type substrate, wherein said induction is a geometric type induction.

A second object of the present invention is a method for differentiating stem cells by using a nichoid-type substrate, preferably towards a neural phenotype.

In a further embodiment, stem cells cultured on a nichoid-type substrate proved to be surprisingly adapted for in-vivo transplants.

### DETAILED DESCRIPTION OF THE INVENTION

Nichoid

In the present description, the term “nichoid” means microscaffolds (or “synthetic niches”), preferably prepared by 2PP technology in the commercially available photoresist SZ2080.

The first description of such microscaffolds is found in M T et al. (Three-dimensional structural niches engineered via two-photon laser polymerization promote stem cell homing. Acta Biomater. 2013; 9(1):4579-84).

Their fabrication is also described in Ovsianikov A, Viertl J, Chichkov B, Oubaha M, MacCraith B, Sakellari I, et al. (Ultra-low shrinkage hybrid photosensitive material for two-photon polymerization microfabrication. ACS Nano 2008; 2:2257-62).

The microscaffolds have also been described in (Ovsianikov A Engineering 3D: Multiphoton processing technologies for biological and tissue engineering applications. Rev Med Devices. 2012; 9: 613-33).

The nichoid consists of an inorganic-organic hybrid sol-gel resin synthesized with silicium (S)-zirconium (Z).

The main components of SZ2080 are methacryloxypropyl trimethoxysilane and zirconium propoxide with the addition of 1% concentration of photoinitiator Irg (Irgacure 369, 2-Benzyl-2-dimethylamino-1-(4-morpholinophenyl)-butanone-1) (Ciba Specialty Chemicals, Basel, Switzerland).

In an embodiment, the overall size of each block of nichoids is 30 μm in height and 450 μm×450 μm in transverse dimensions.

The spacing between the blocks of nichoids is 15 μm.

Every block consists of 25 repeating nichoid unities (5×5), 30 μm in height and 90×90 μm in transverse dimensions, consisting of a grid of interconnected lines, with a graduated spacing between 10 and 30 μm transversely and a uniform spacing of 15 μm vertically.

Every nichoid, as well as every block of 5×5 nichoids, is surrounded by four outer confinement walls consisting of horizontal lines spaced 5 μm apart, resulting in a 1 μm gap.

A “nichoid” must be understood as a structure, which artificially reproduces the environment of the stem cell niches.

Therefore, for the purposes of the patent application, reference can also be made to the term “nichoid” using the term “synthetic niche matrix” or “synthetic niche substrate”.

In accordance with a first object, the present invention describes a method for inducing pluripotency in cells by using a nichoid-type substrate.

In a particular aspect of the present invention, said induction is a geometric type induction.

The pluripotency induction represents the expression of genes which bring an adult cell of any type (a stem cell or not) back to the staminality state of embryonic type, referred to as pluripotency.

More particularly, the pluripotency induction is the increase in the expression of pluripotency genes.

Even more in detail, the pluripotency induction is to be understood as the increase in the expression of pluripotency genes, which comprise the genes Nanog, Sox2 and Oct4.

Therefore, the pluripotency induction is a highly different phenomenon from the maintenance of staminality, by which it is meant the expression of the genes, which maintain an adult stem cell in such conditions, preventing it from differentiating, i.e. maturing towards a different phenotype.

In particular, said method comprises the steps of:

a) plating the cells on a nichoid-type substrate;

b) allowing said cultured cells to proliferate for a certain period of time.

As regards step b), the proliferation is carried out for a period of time between about 1 and 10 days, which period of time is preferably about 7 days.

In an aspect of the invention, after the proliferation step b), the cells are detached from the substrate (step c).

The detachment of the cells from said nichoid is preferably achieved with one sodium citrate solution.

Preferably, such a solution has a concentration of sodium citrate of 1-20 mM.

In an aspect, such a solution comprises 0.135 M KCl and 0.015 M sodium citrate.

Advantageously, it has been seen that even once the cells have been detached from the nichoid, they maintain the organization given by the nichoid.

Once the cells have been detached from the substrate, they are cultured (step d).

In a preferred aspect of the invention, the cells are cultured in suspension or under adhesion.

For the purposes of the present invention, the cells subjected to pluripotency induction can be stem cells or non-stem cells.

In a preferred aspect, said cells are stem cells.

In an even more preferred aspect, said stem cells are chosen from the group, which comprises: adult, embryonic, cordonal, placental or fetal stem cells.

In an aspect of the present invention, such adult stem cells are neural progenitors or are mesenchymal cells.

In a particular aspect, such mesenchymal cells are cells derived from human adipose tissue.

In an even more preferred aspect, such cells are Er-NPCs (Erythropoietin-releasing Neural Precursor Cells).

In a further embodiment, a method of differentiating stem cells is described, which comprises using a nichoid.

In an aspect of the present invention, such a method does not require to use any cell adhesion-promoting substrate.

In a preferred aspect, the cells employed in the method of the present invention are neural progenitor cells, which, in an even more preferred aspect, are Er-NPCs (Erythropoietin-releasing Neural Precursor Cells).

For the purposes of the present invention, such a method comprises the steps of:
i) plating said cells on a nichoid-type substrate,
ii) replacing the culture medium with a medium which comprises serum,
iii) harvesting the differentiated cells.

In particular, in step i) cells are plated in the presence of a culture medium, which does not comprise serum.

Such a culture medium can be represented by 10 mg/mL NSC medium+ bFGF.

Cells are preferably plated at a concentration of about 1.5×10⁴ cells/cm².

More in detail, such cells are plated after being mechanically dissociated.

As regards step ii), this includes the replacement with a culture medium which comprises serum.

In particular, step ii) is carried out after about 3 days.

As described above, step i) is carried out in the absence of a cell adhesion-promoting substrate; an example of such a substrate is represented, for example, by vitronectin or Matrigel™.

In a further aspect of the present invention, the differentiation method described above allows to obtain neuronal cells.

The present invention also relates to the medical use of said neuronal cells.

In particular, such cells can be used for medical therapeutic use.

More particularly, the medical use is described for the treatment of neurodegenerative diseases.

Even more particularly, such cells can be employed for use in intracerebral or intraspinal or intravenous transplantation.

The following examples serve to better understand the invention and are not to be considered as limiting the invention, the scope of which is defined by the following claims.

### Materials and Methods

### Cells

In the present invention, Er-NPCs were used, a subclass of neural progenitors derived from the subventricular zone, capable of surviving for 6 hours after the donor death. They exhibit greater neural differentiation than the cells taken from the same region immediately after death.

These cells are referred to as erythropoietin-releasing neural precursor cells (Er-NPCs) since they mainly differentiate into neurons, show the activation of the hypoxia-inducible factor 1 and MAPK, and express both erythropoietin (EPO) and the receptor thereof (EPO-R). Er-NPCs favor the preservation of axonal myelin and strongly promote regrowth through the lesion site of the monoaminergic and catecholaminergic fibers, which reach the caudal parts of the injured cord. When Er-NPCs are assayed in a proliferation test, they can increase in number. They seem floating neurospheres, which do not adhere to the substrate. Given their non-adherent growth, Er-NPCs can be cultured without distinction on slide or plastic. In a differentiation test, the plated cells cannot increase in number, but differentiate into neuronal cells and grow adherent to the substrate. In order to enable this adhesion, using a biological substrate is always required. In this case, since the cells are adherent, they were plated on the slide (positive control for the differentiation test).

### Medium and Substrates

Medium neural stem cells (NSC medium): Neurobasal® Medium (GIBCO®, Life Technologies Italia, Monza, Italy) containing 2% B-27® supplement, 2% L-Glutamine (Euroclone, Pero, MI, Italy), 1% penicillin and streptomycin (Euroclone, Pero, MI, Italy), b-FGF (human recombinant, 20 ng/mL, Peprotech, Rocky Hill, N.J., USA, or Upstate Biotechnology, Lake Placid, N.Y., USA) and h-EGF (human recombinant, 20 ng/mL; Peprotech).

Differentiation medium 1: NSC medium with β-FGF (10 ng/mL) without h-EGF.

Differentiation medium 2: NSC medium without β-FGF and h-EGF with 1% fetal bovine serum (FBS).

In order to allow the adhesion of Er-NPCs in the differentiation dosage, Matrigel™ is used as a biological support. Matrigel™ is the trade name for a gelatinous protein mixture secreted by Engelbreth-Holm-Swarm (EHS) mouse sarcoma cells. In order to create a biological support for cells, a small volume of liquid Matrigel™ refrigerated (4° C.) is delivered to a well with a slide therein. When it is incubated a 37° C. (body temperature), its proteins polymerize (solidify) yielding a gel which covers the slide surface. Thick gels cause the cells to migrate from the surface of the gels to their interior. The ability of this biological support to stimulate the complex cellular behavior is a consequence of the heterogeneous composition thereof. The main components are structural proteins such as laminin, nidogen, collagen and heparan sulfate proteoglycans which have adhesive peptide sequences as under physiological conditions.

### Antibodies

For immunofluorescence assays, the antibodies were used at the following dilution:

Anti-Nestin (monocl.1: 100; Millipore; anti-mouse), staminality marker in cytoskeleton.

Anti-microtubule-associated protein 2 (MAP2; 1: 200; Millipore; anti-rabbit), highlights mature neurons and the cellular localization thereof is within the cytoskeleton.

Anti-erythropoietin (1: 200; GeneTex; anti-mouse and human), is a glycoprotein cytokine secreted by the kidney in response to cell hypoxia and the cellular localization thereof is in the cytoplasm.

Anti-beta-tubulin III (TUJ1; 1: 400; Millipore; anti-mouse), highlights immature neurons and the cellular localization thereof is within the cytoskeleton.

For Western blot analysis, the antibodies are as follows:

Anti-SRY (sex-determining region Y)-box 2 (monocl.1: 500; Sigma; anti-rabbit): also known as SOX2, an essential transcription factor for maintaining self-renewal, or pluripotency, of undifferentiated embryonic stem cells.

Sox2 is a pluripotency marker of all stem cells; such a factor is not to be confused with marker Sox1, which is instead a marker for maintaining staminality in cells deriving from the ectodermal leaf.

Anti-Oct4 (monocl.1:500; Sigma; anti-rabbit): involved in the self-renewal of undifferentiated embryonic stem cells. As such, it is often used as a marker for undifferentiated cells.

Anti-erythropoietin (monocl.1: 200; GeneTex; anti-rabbit): a glycoprotein cytokine secreted from the kidney in response to cell hypoxia.

Anti-erythropoietin receptor (monocl. 1:200; Millipore; anti-rabbit).

Anti-β-actin (1:500; Millipore; anti-mouse).

Secondary antibodies are:
immunofluorescence:
Alexa fluor 543 goat anti-rabbit IgG (1: 1000; Invitrogen, Life Technologies Italia, Monza, Italy).
Alexa fluor 543 goat anti-mouse IgG (1: 1000; Invitrogen, Life Technologies Italia, Monza, Italy).
Alexa fluor 488 goat anti-rabbit IgG (1: 1000; Invitrogen, Life Technologies Italia, Monza, Italy).
Alexa fluor 488 goat anti-mouse IgG (1: 1000; Invitrogen, Life Technologies Italia, Monza, Italy).
Western blot:
Anti-rabbit (1: 1000; Invitrogen, Life Technologies Italia, Monza, Italy).
Anti-mouse (1: 1000; Invitrogen, Life Technologies Italia, Monza, Italy).

### EXPERIMENTAL DATA

### Example 1

### Er-NPCs Isolation and Characterization

Post-mortem neural precursors were obtained from 2-month-old CD1 mice and CC57BL/6 57 black mice, 6 hours after the animals' death. The animals were held under standard conditions for at least 3 days prior to the trials (22±2° C., 65% humidity and artificial light between 8:00 am and 8:00 pm).

Mice were anesthetized by intraperitoneal injection of 4% cloral hydrate (0.1 mL/10 g body weight) and sacrificed by cervical dislocation. The corpses were kept for 6 hours at room temperature (25° C.). After this period, their brain was removed and the cells isolated from the SVZ (subventricular zone of the lateral ventricle). In short, the protocol was:

a) Transferring the dissected tissue into a phosphate buffer solution containing penicillin, streptomycin (each with a concentration of 100 U/mL) (Invitrogen, San Diego, Calif., USA) and glucose (0.6%) at 4° C. until the end of the dissection;
b) Transferring the tissue into a Earl's balanced saline solution (EBSS) (Sigma-Aldrich, St. Louis, Mo., USA) containing 1 mg/mL papain (27 U/mg, Worthington DBA, Lakewood, N.J., USA), 0.2 mg/mL cysteine (Sigma-Aldrich) and 0.2 mg/mL EDTA (Sigma-Aldrich) for performing the enzymatic dissociation;
c) Incubating for 45 minutes a 37° C. on a rocking platform.
d) Centrifuging the tissues at 123 g and discarding the supernatant.
e) Resuspending the pellet in 1 mL EBSS and mechanically dissociating it using an aerosol resistant tip (1000 μL Gilson pipette). Cells were resuspended in 10 mL EBSS.
f) Centrifuging at 123 g for 10 minutes, discarding the supernatant and resuspending the pellets in 200 μL EBSS.
g) Resuspending the pellet in 1 mL EBSS and mechanically dissociating it using an aerosol resistant tip (200 μL Gilson pipette). Cells were resuspended in 10 mL EBSS.
h) Centrifuging at 123 g for 10 minutes, discarding the supernatant and resuspending the pellets in NSC medium.
i) Plating the cells at 3500 cells/cm² in the appropriate medium volume in a 25 cm² flask, at 37° C. in a humidified atmosphere with 5% CO₂.

### Example 2

### Cultured Er-NPCs

Er-NPCs were plated in the growth medium containing b-FGF and h-EGF. After one week, in the absence of serum, these cells originated floating neurospheres in culture with a diameter of 75/100 μm. Tripan blue exclusion was used to evaluate the total number of viable cells. The thus formed spheroids were harvested by centrifugation (10 minutes at 123 g), mechanically dissociated by pipetting in a single cell suspension and re-plated on average at a density of 10,000 cells/cm². This procedure was repeated every 4-5 days.

### Example 3

### Neuronal Differentiation of Er-NPCs

Er-NPCs, in order to check the multipotency of neural stem cells, were subjected to in vitro differentiation. The neurospheres were mechanically dissociated and seeded on a glass coverslip with Matrigel™ coating (diameter 10 mm) in the presence of bFGF (10 ng/mL). After 48 hours, the cells were moved to the differentiation medium where bFGF was replaced with FBS (1% of the total volume of the medium) for 5 days. Er-NPCs attached to the dish and differentiated into the three cells types found in adult CNS: neurons, astrocytes and oligodendrocytes in a typical cellular stretching ratio.

### Example 4

### Preparation of the Nichoid and Cell Culture

The 2PP patterned substrates and the glass controls were placed within a multiwell plate with 24 wells.

In order to culture neural precursors within the nichoid, the thus formed neurospheres in culture as indicated in Example 2 were:

a) harvested;
b) harvested by centrifugation (10 min at 123 g);
c) mechanically dissociated by pipetting to a single cell suspension;
d) resuspended in 30 μL of medium containing bFGF (10 ng/mL) and EGF (10 ng/mL);
e) plated on a nichoid-type substrate;
f) maintained for 1 hour at 37° C., 5% 002, for allowing the cells to enter into the niches;
g) added with 500 μL of the same medium used in d).

For the neuronal differentiation of Er-NPCs within the nichoid, the thus formed neurospheres in culture were:

a) harvested;
b) harvested by centrifugation (10 minutes at 123 g);
c) mechanically dissociated by pipetting to a single cell suspension;
d) resuspended in 30 μL of differentiation medium 1 (10 ng/mL NSC medium+bFGF);
e) plated on a nichoid-type substrate;
f) maintained for 1 hour at 37° C., 5% 002, for allowing the cells to drop into the niche;
g) 500 μL of differentiation medium 1 were added to the multiwell plate;
h) after 48 hours, the cells were moved to differentiation medium 2 (NSC medium+2% FBS) for at least 5 days.

### Example 5

### Detachment

In order to identify a detachment procedure for counting the cells grown within the nichoid, different solutions were compared in one dosage on slides. The following methods were tested:
Mechanical dissociation (positive control)
Tripsin-EDTA 0.05/0.02%
Tripsin-EDTA 0.02/0.01%
Tripsin-EDTA 0.01/0.005%
10 mM EDTA
Accutase®: enzymes accutases in Dulbecco's phosphate buffered saline (0.2 g/L KCl, 0.2 g/L KH₂PO₄, 8 g/L NaCl and 1.15 g/L Na₂HPO₄) containing 0.5 mM EDTA⋅4Na and 3 mg/L phenol red
Citric saline solution (described above)

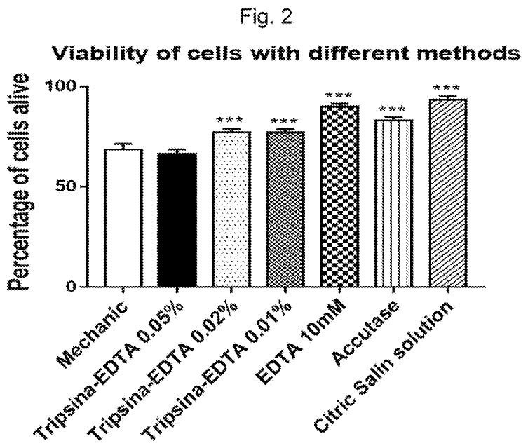

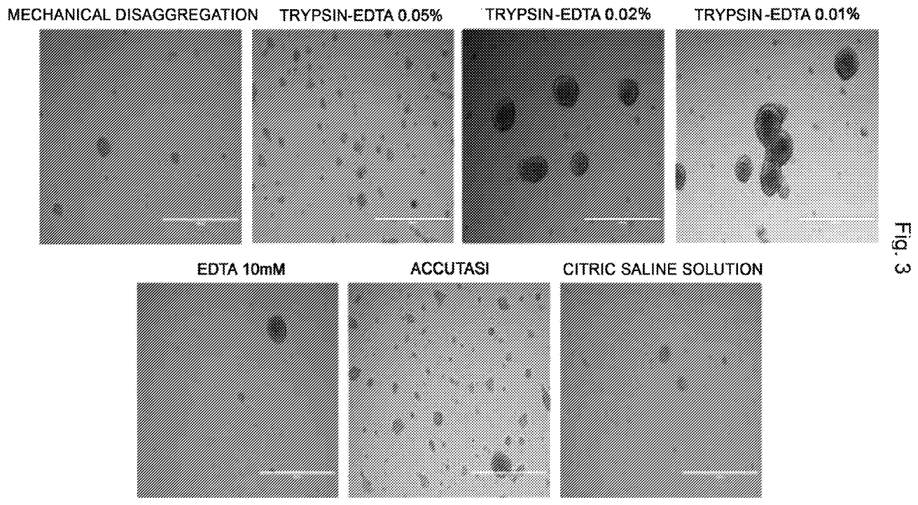

The cells incubated with the various solutions indicated above were incubated at 37° C., 5% CO₂, for 10 minutes except for the treatment with citric saline solution, which was incubated under the same conditions for 4 minutes. The number of detached cells was counted with a hemocytometer by a trypan blue exclusion method. Data are shown in FIG. 2.

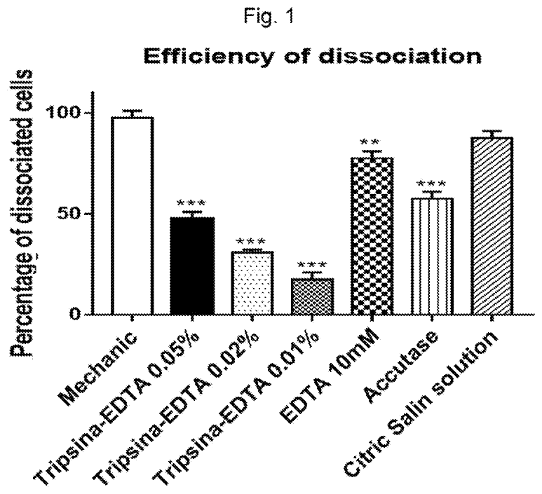

The plot in FIG. 1 shows the efficiency of dissociations using different methods of detachment. Using sodium citrate solution or 10 mM EDTA favors the cell dissociation to a much greater extent than all other conditions. The two methods originate a number of dissociated cells (80%) similar to the mechanical procedure, used as a control (90%).

FIG. 2 shows that the number of dead cells is significantly low for the citric saline solution and 10 mM EDTA. Moreover, the treatment with sodium citrate solution and 10 mM EDTA allows a better preservation of cell viability.

Alternative methods to mechanical disaggregation are:
Citric saline solution
10 mM EDTA

The cell re-plating showed, in all cases, that living cells started to form neurospheres (FIG. 3). The large size of the spheroid formed in the case of treatments with trypsin EDTA and Accutase could be due to inefficient dissociation (FIG. 3).

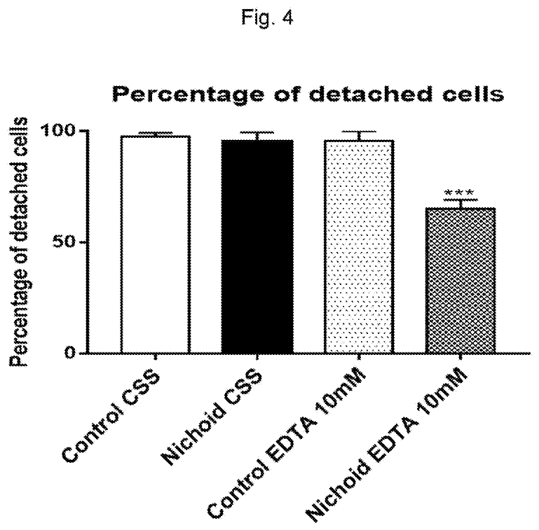

The two detachment procedures selected were then utilized on the nichoid. 1×10⁴ Er-NPCs were plated on the nichoid and allowed to grow within the niches for 1 week. The culture medium was removed and the cells were alternatively treated with two detachment solutions, 10 mM EDTA and/or CSS. After 4 and 10 minutes, indeed, the cells were not detached. After 50 minutes, the cells treated with citric saline solution were completely detached. The nichoid treated with 10 mM EDTA, however, showed 40% of cells still adhering (FIG. 4).

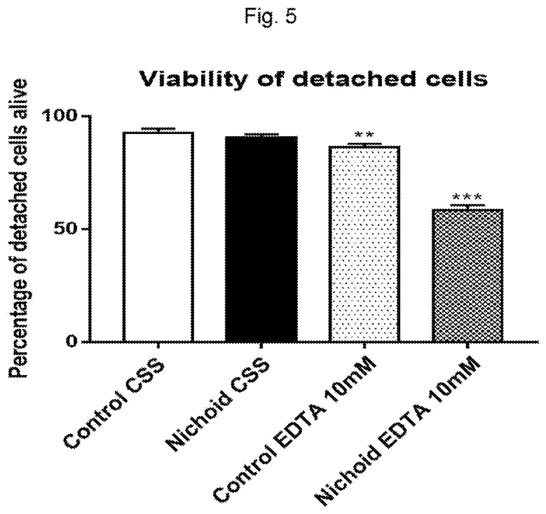

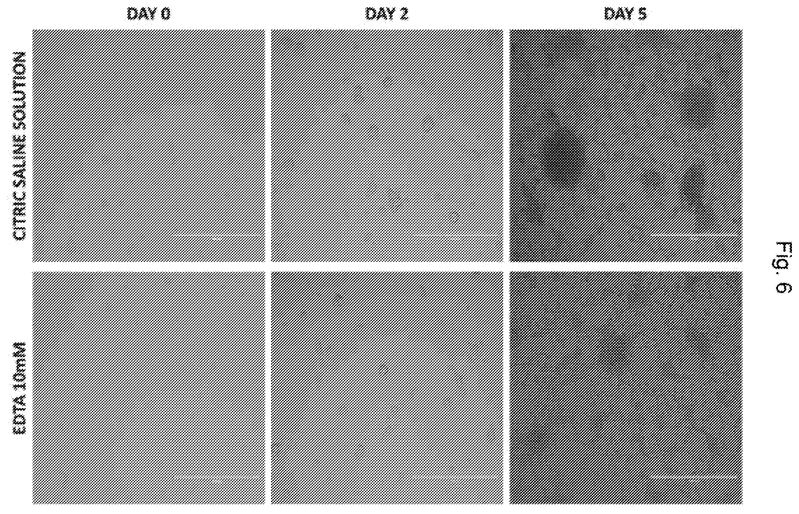

In the CSS treatment, the viable cells were more than 90% of detached cells, instead they were 60% in the sample treated with 10 mM EDTA (FIG. 5). Once the cells were detached from the nichoids, they were plated under floating conditions with NSC medium, for evaluating whether they were still capable of reforming spheroids. Spheroid reformation has successfully occurred since the second day. On day 5, Er-NPCs detached from the nichoid form spheroids with a diameter between 75 and 100 μm, similar to that formed under standard floating conditions (data not shown).

### Example 6

### Proliferation Assay

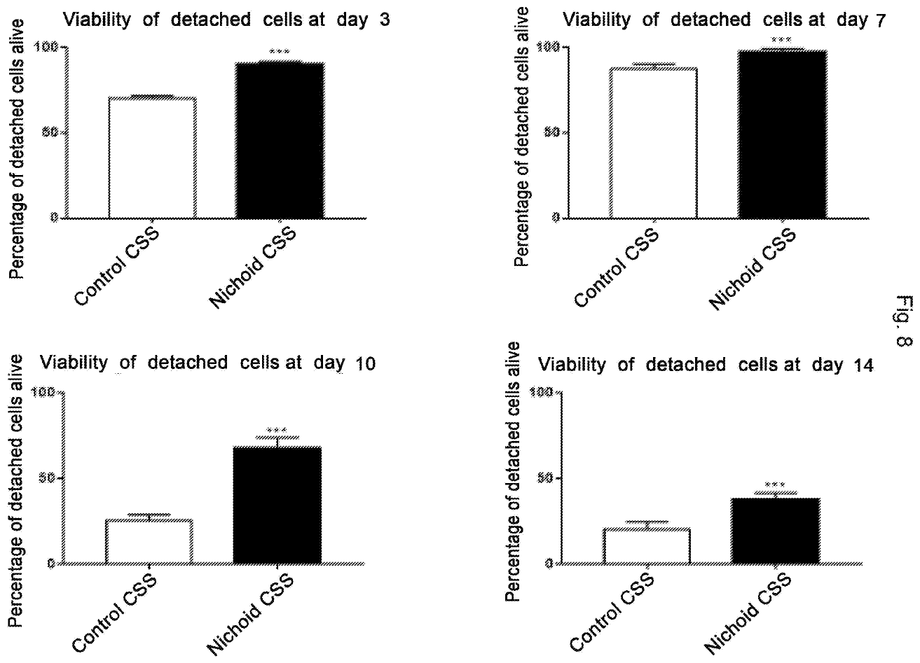

1×10⁴ cells were plated and grown for 3, 7, 10 and 14 days after plating. In each time point, the cells were detached from the CSS treatment as described in Example 5, and the number of cells was determined by the blue trypan exclusion method. Every condition was double-plated and counted by two different blinded operators. As a control, the same amount of Er-NPCs was plated under fluctuating conditions in the same growth medium. The total cell number is in FIG. 7.

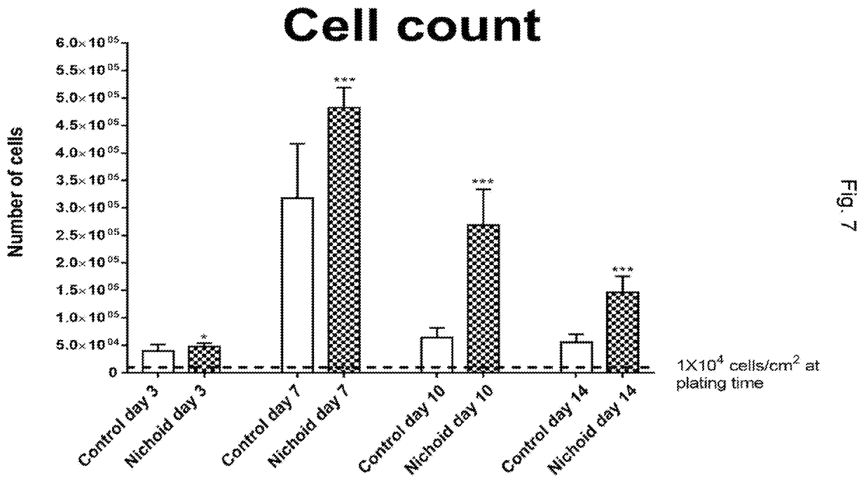

The growth capacity of Er-NPCs is always greater in the niches than under fluctuating conditions, used here as a positive control (FIGS. 6, 7, 8). The time required for the cell detachment on days 10 and 14 from the nichoids was greater than that on day 7, 80 minutes for day 10 and 120 minutes for day 14, respectively. This is probably due to the growing number of cells and the resulting greater adhesion with the nichoid and between the same layers of cells (FIGS. 7, 8, 9, 10).

The highest number of mortality from the 10th day onwards is probably due to the fact that Er-NPCs are normally passed after 7 days, as shown by the trial (if not passed, they start to die) and to the possible lack of nutrients after 10 days.

### Example 7

### Characterization of the Growth of Er-NPCs within the Nichoid

In order to investigate the expression of EPO and Nestin, the immunofluorescence analysis was performed on Er-NPCs grown for 7 days within the nichoid with respect to the control. Moreover, the possible coexpression of EPO and TUJ was studied.

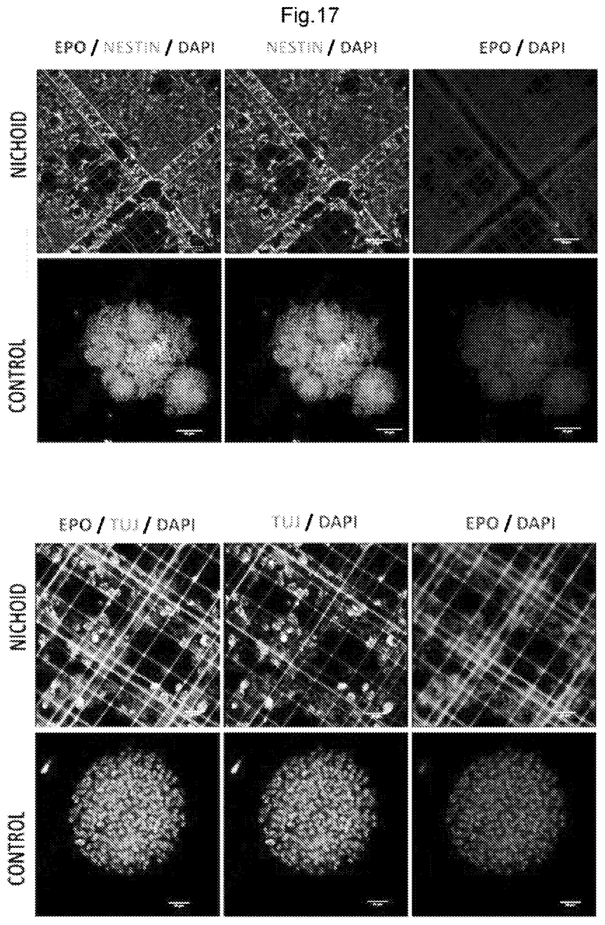

From the confocal images, it can be seen how the cells of the nichoid maintain the ability to express EPO, Nestin and TUJ, already observed at baseline in cells grown under standard fluctuating conditions (control) (FIG. 17).

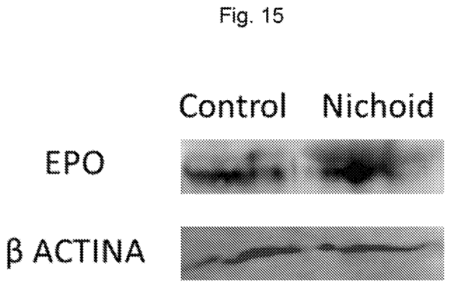

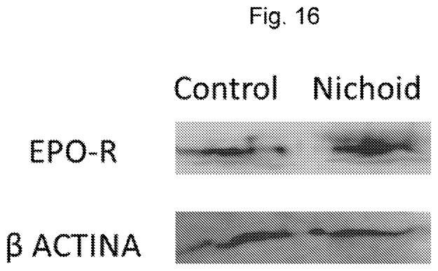

The expression of EPO and EPO-R was also studied by Western blot analysis in Er-NPCs grown within the nichoid for 7 days with respect to standard fluctuating conditions in NSC medium. The cells were lysed in RIPA buffer, the proteins quantified, and 50 μg of total proteins were loaded into SDS-PAGE under reducing conditions (final concentration of 2-β-mercapthoethanol of 5%). The expression of the investigated factors is not significantly different from the standard floating conditions (FIGS. 15 and 16).

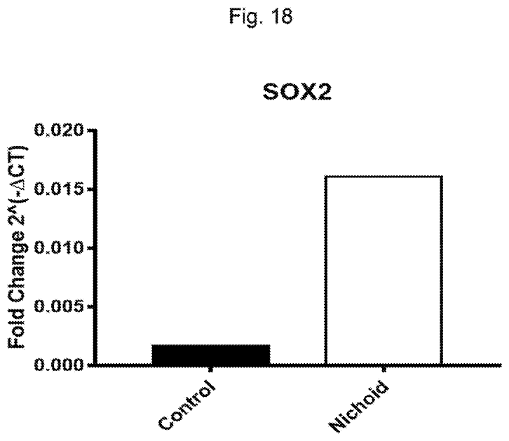

From the acquired images, it was also possible to investigate the distribution of specific markers with respect to the Z axis within the nichoid (FIG. 18). Nestin and EPO in Er-NPCs, grown under floating conditions, are expressed in the cytoplasm of the cells contained in the neurospheres (Schiffer, 2006). On the other hand, Tuj seems to be mainly expressed by those cells found in the outermost part of the neurospheres (Jirásek, 2009). It can be seen how EPO follows the distribution of DAPI while Nestin, by highlighting the cell extensions, is distributed more over the entire structure (FIG. 18 a, b, c). Accordingly, the cells developed a complex multi-branch connection within the nichoid. The expression of EPO in the second immunofluorescence, with TUJ, confirms what was seen in the previous trial (FIG. 18 d, e, f).

In this immunofluorescence, we can see how the cells are more concentrated in the central layers of the structure where they form an aggregate. Tuj is expressed more by those cells localized in the outermost part of this aggregate.

### Example 8

Expression of Staminality Markers

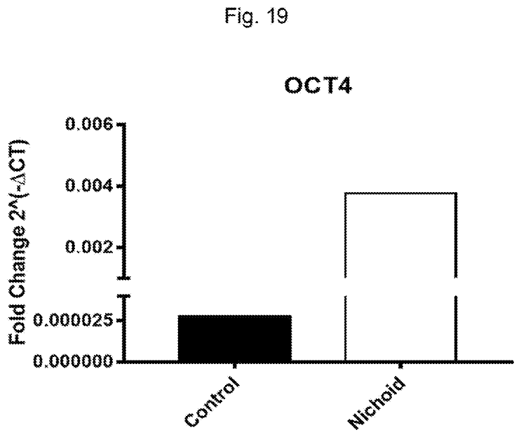

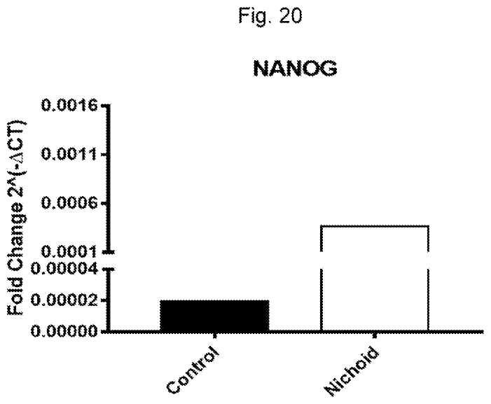

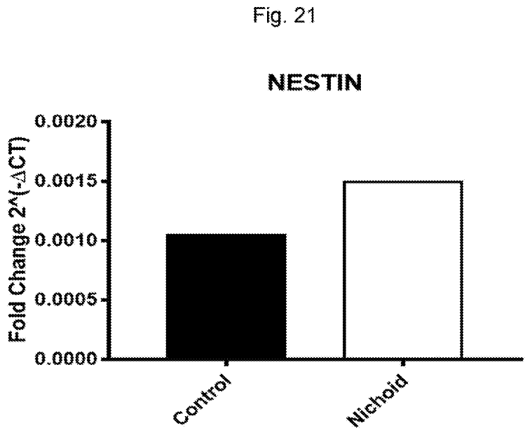

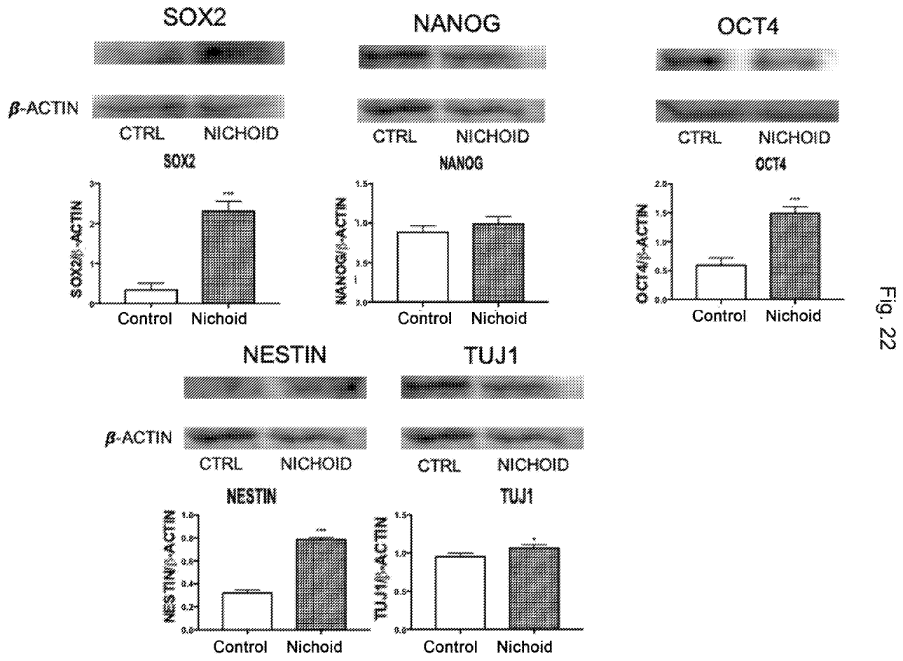

An mRNA analysis of Er-NPCs grown within the nichoids for one week was performed with respect to the control conditions (cells cultured in NSC medium in suspension on a slide). The expression of Sox2, Oct4, Nanog and Nestin was studied by real-time RT-PCR. In FIG. 19, we can see that we have a large increase in the level of SOX2 mRNA in Er-NPCs grown (8 times higher) in the nichoid with respect to the fluctuating control conditions. FIG. 20 shows that the level of OCT4 mRNA in Er-NPCs grown within the nichoid is 160 times higher than in the control. In FIG. 21, we can see that the level of NANOG mRNA has increased by 20 times compared to the expanded cells in the nichoid with respect to the control.

Nestin is an intermediate-stranded protein expressed in dividing cells during the early stages of development in the central nervous system, peripheral nervous system and myogenic tissues and others. At the time of differentiation, nestin is down-regulated (Matsuda, 2013). FIG. 22 shows that there is an increase in the level of Nestin mRNA in the nichoid with respect to the control.

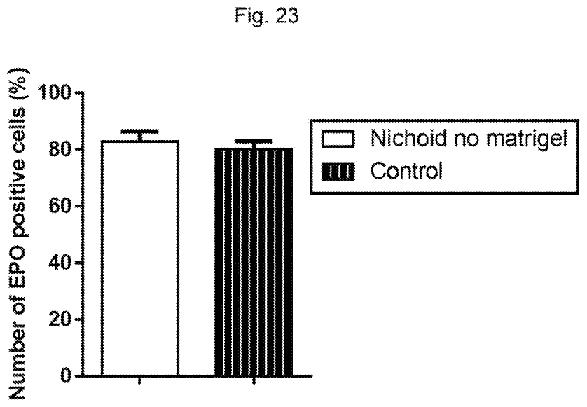

The expression of Sox2, Oct4, Nanog, nestin and TUJ1 was also studied by Western blot analysis in Er-NPCs grown within the nichoid for 7 days with respect to standard fluctuating conditions in NSC medium (Gritti, 2002; Marfia, 2011). The cells were lysed in RIPA buffer, the proteins quantified, and 50 μg of total proteins were loaded into SDS-PAGE under reducing conditions (final concentration of 2-β-mercapthoethanol of 5%). The expression of the investigated factors is not significantly different from the standard floating conditions (FIG. 23).

### Example 9

### Differentiation of Er-NPCs within the Nichoid

The differentiation of Er-NPCs is normally achieved using a biological matrix (Matrigel™). In fact, this matrix is intended to allow the adhesion and avoid the cell death. The differentiation is obtained by plating 1.5×10⁴ cells/cm². In this case, the number of cells does not increase, unlike proliferation, but it differentiates in a mixed population of neurons and glial cells. The time schedule followed during the differentiation has three fundamental stages.
Day 1, in which the neurospheres are mechanically dissociated and the single cells are plated in the presence of bFGF (10 ng/mL) and adhesive substrate. At this point, the cells start adhering.
Day 3, when the cell medium is changed to a serum containing a fetal bovine serum (2% FBS).
Day 8, when the cells are differentiated in a mixed population of neuronal and glial cells.

In order to understand if the presence of Matrigel™ was necessary inside the nichoid, the differentiated cells on the plate with Matrigel™ (positive control) were compared with the nichoid with Matrigel™ or with the nichoid without any organic substrate (Matrigel™). The cells were differentiated using the procedure just described. The number of plated cells was 1.5×10⁴ cells/cm². Using a digital optical microscope (EVOS), we counted the adhered cells and were able to determine the number of adherent cells. The results show that 79.28% of Er-NPCs adhere within the nichoid without Matrigel™, in comparison to 86.16% of cells under control conditions (flat slide coated with Matrigel™). Only 31.12% of cells adheres if the nichoid is coated with Matrigel™. This suggests how the cell differentiation is possible within the nichoid without the use of Matrigel™.

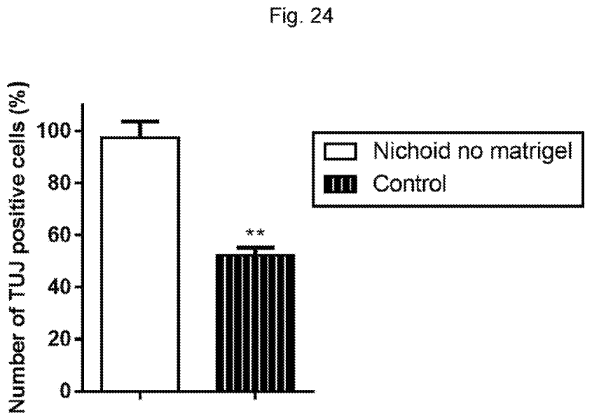

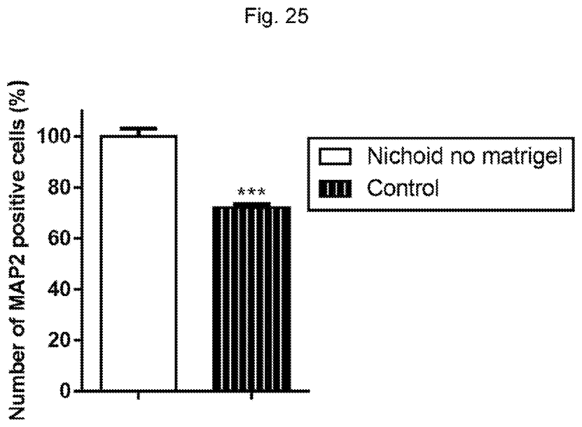

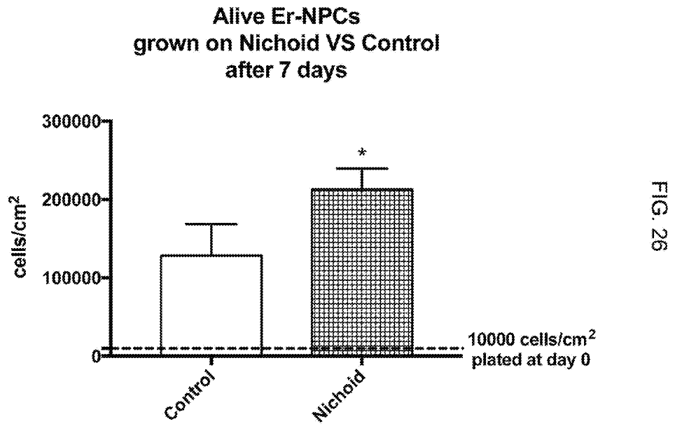

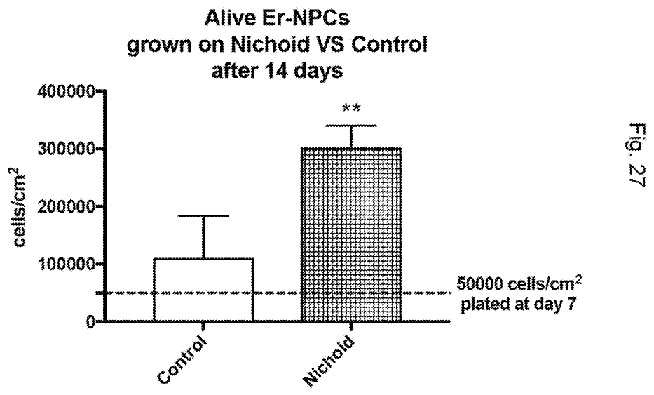

In order to study the differentiation ability of Er-NPCs without Matrigel™ within the nichoid, with respect to the control, we performed an immunofluorescence with TUJ (marker of neural precursors) (Baldassaro, 2013) and EPO. For this immunofluorescence analysis, all the analyzed instances were plated with an initial number of 1.5×10⁴ cells/cm². In the nichoid, the cells seem to express both markers with greater intensity than the control. As demonstrated in the previous trial, the nichoid cells continue to express EPO at a level, which is equal to the control (FIG. 24), instead the TUJ-positive cells significantly increased with respect to the control (FIG. 25).

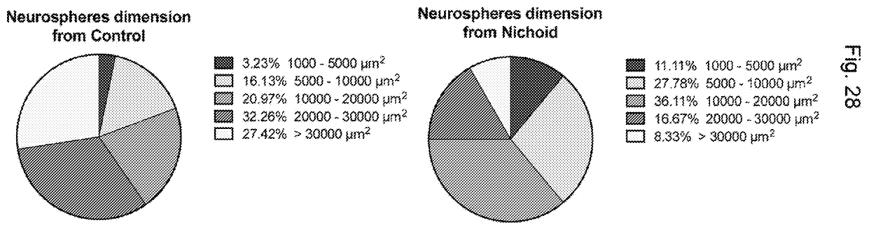

In order to confirm the ability of Er-NPCs to differentiate in the neuron after growth in the nichoid, we performed an immunofluorescence for Map2 (marker of mature neurons) and Nestin (staminality marker) to demonstrate the co-expression of both markers. For this immunofluorescence analysis, all the analyzed instances were plated with an initial number of 1.5×10⁴ cells/cm². Map2-positive cells are also Nestin-positive. The plots show a higher level of Nestin and Map2 in differentiated Er-NPCs within the control of the nichoid (FIG. 26). This immunofluorescence confirms the data shown in the previous Figures.

### Example 10

### In Vivo Transplantation

Er-NPCs physiologically expressing GFP and cultured for one week within the nichoids from which they were detached, using the CSS method, were then used for in vivo transplantation.

Parkinsonism was induced by intraperitoneal administration of 1-methy-4-phenyl-1,2,3,6-tetrahydropyridine (MPTP) in C57/bl mice following the acute paradigm with a small modification.

The animals were administered with a double dose of MPTP hydrochloride:

1. intraperitoneally, an injection of MPTP (36 mg/kg)
2. after 7 days, i.p. injection of MPTP (20 mg/kg)

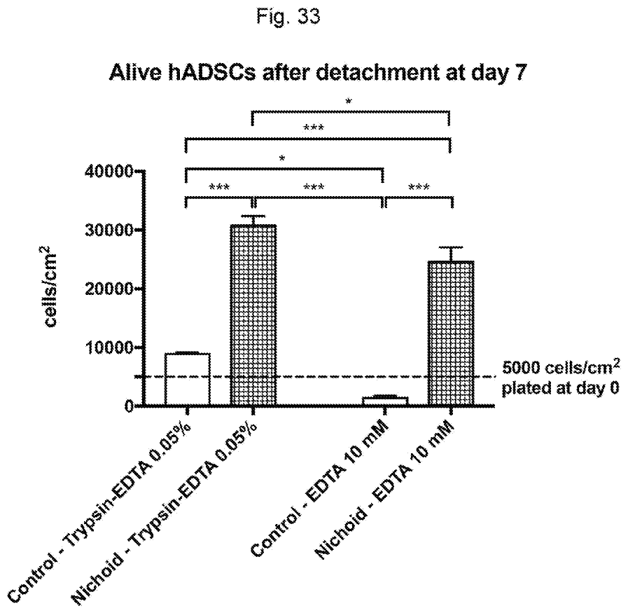

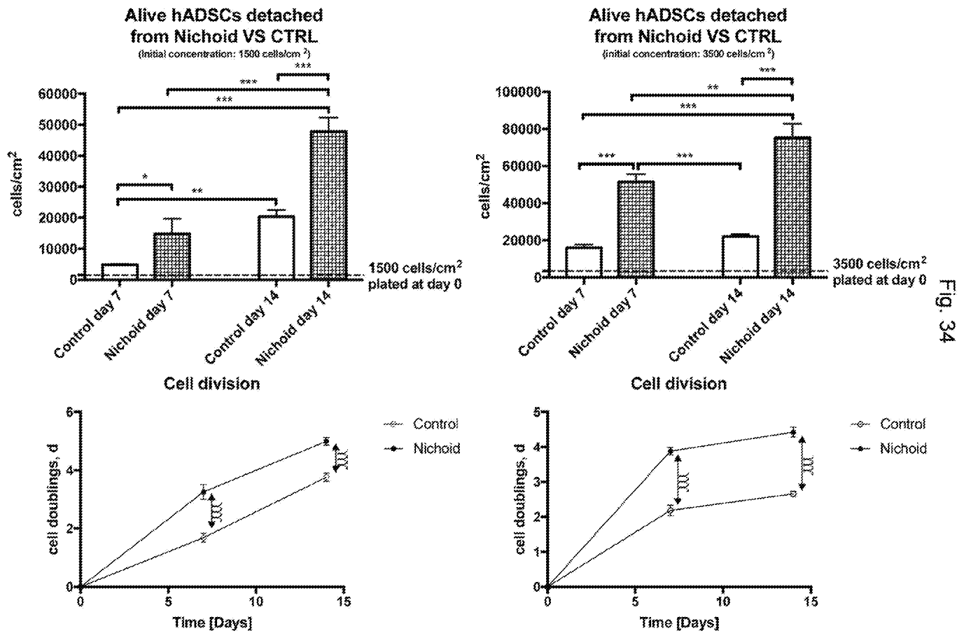

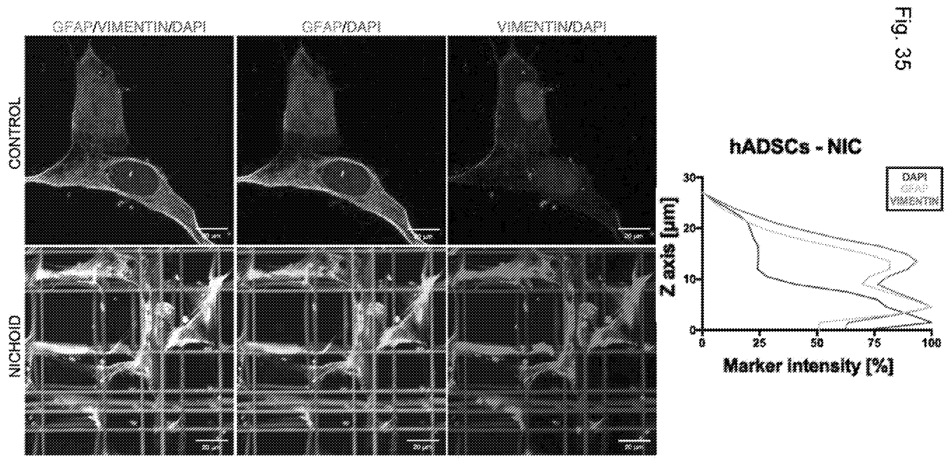

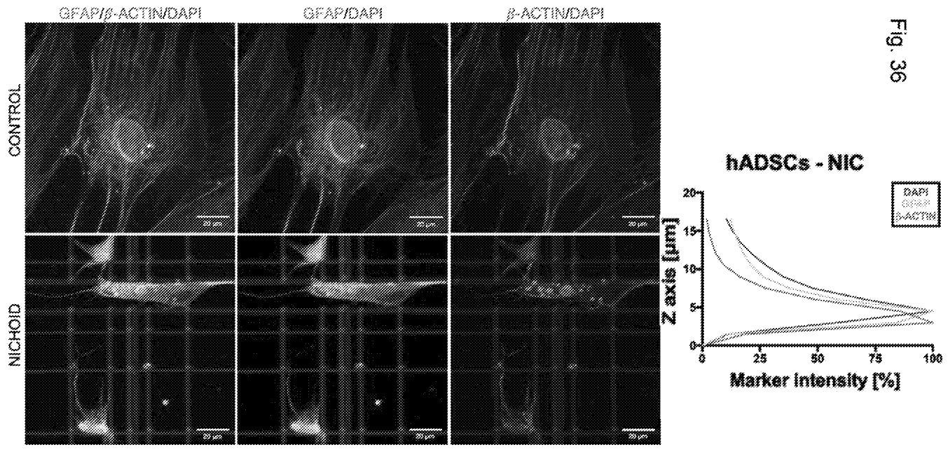

5×10⁴ cells/mL (5 μL) of GFP Er-NPCs inside the wells (3) or inside the nichoids (3) were transplanted to a group of animals, according to the following stereotaxic coordinates in relation to the bregma: 0.1 mm posterior, 2.4 mm dorsal medial-lateral and 3.6 mm at the level of the left striatum (Cui, 2010). All animal management was fully compliant with the good zootechnical practices defined by the Italian Guidelines for laboratory animals, which in turn comply with the European Community Directive dd. September 2010 (2010/63/EU); the work was approved by the review committee of the University of Milan. The animals survived the transplantation. The data on the therapeutic potential of the transplanted cells confirmed the functional recovery of the forelimbs as in the case of the control cells (FIG. 33).

### Example 11

### Expression of Staminality Markers in Neural Precursors Grown within the Nichoid

The investigation on staminality markers such as SOX2, OCT4, NANOG and NESTIN from neuronal precursors grown within the nichoid for 7 days was performed evaluating the expression of both mRNA by real-time RT-PCR and proteins by Western blotting. The maintenance of neural precursors within the nichoid determines the increase of all targets investigated (FIGS. 19, 20, 21, 22 and 23).

In order to check that the above mRNAs actually yield the corresponding proteins during the transcription step, a further analysis was carried out with the Western blot technique together with TUJ1 which was also seen expressed by immunofluorescence analysis.

This analysis was performed with Er-NPCs grown for 7 days within the nichoid with respect to standard fluctuating conditions in NSC medium with 1×10⁴ cells/cm² as an initial concentration. The cells were lysed in RIPA buffer, the proteins quantified, and 50 μg of total proteins were loaded into SDS-PAGE under reducing conditions (final concentration of 2-β-mercapthoethanol of 5%) (Carelli et al., 2015a). The expression of SOX2, OCT4, NANOG, NESTIN and TUJ1 was evaluated using specific polyclonal antibodies (see Materials and methods for further details). β-actin (42 kDa) was used as a load control (Chen and Xu, 2015). The expression of the investigated factors is significantly induced in the cells grown within the nichoid with respect to those under standard fluctuating conditions (FIG. 23).

### Example 12

Do Er-NPCs Maintain Memory after Growth within the Nichoid?

The purpose of this experimental section was to verify whether Er-NPCs detached from the nichoid after one-week growth were capable of forming neurospheres and maintaining the expression of specific markers.

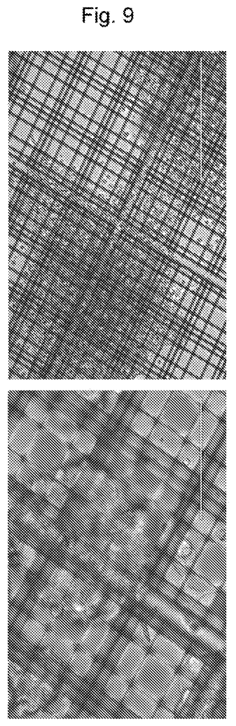

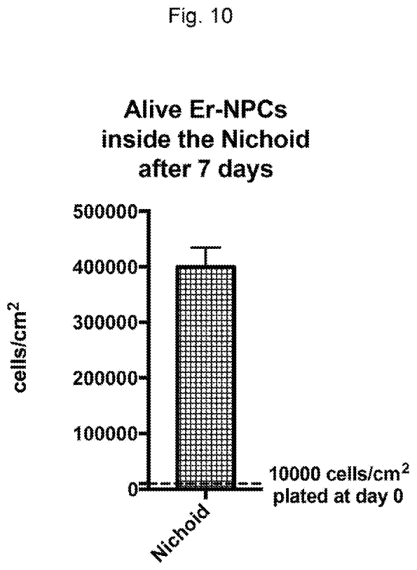

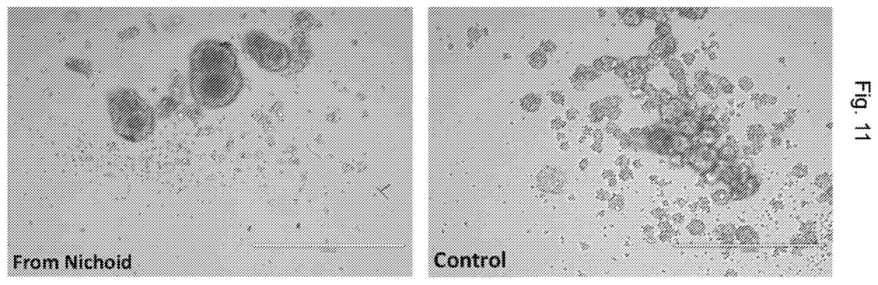

First of all, 1×10⁴ cells/cm² were plated on the nichoid and allowed to grow and, as expected, they formed, after 3 days, new neurospheres, and on day 7, the pad of cells (FIGS. 9 and 10). They were then detached with CSS, dissociated, counted (FIG. 10) and plated again in a new multiwell, under normal fluctuating culture conditions in the presence of NSC medium+bFGF (10 ng/mL)+EGF (10 ng/mL).

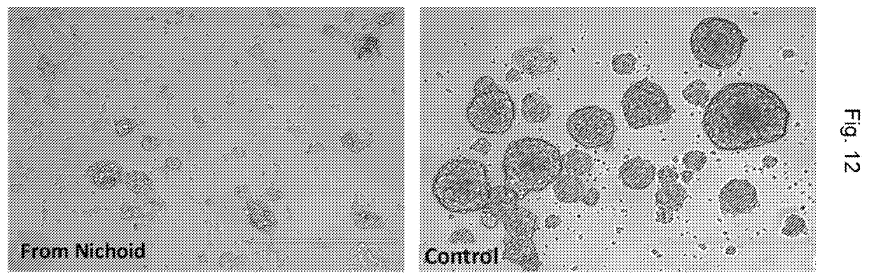

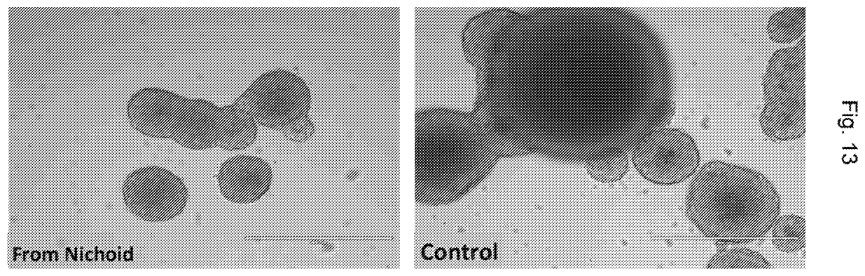

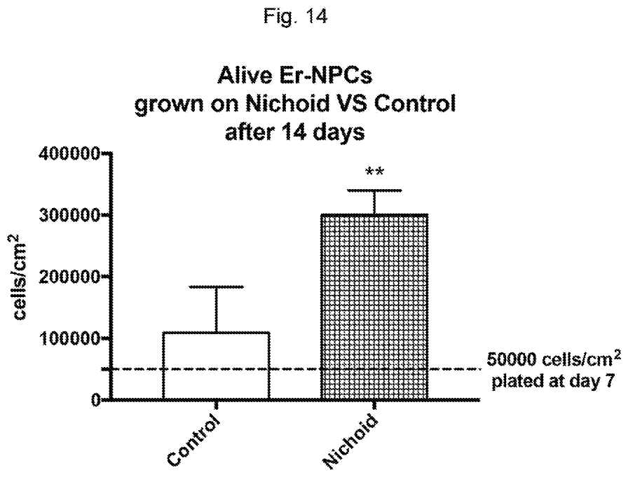

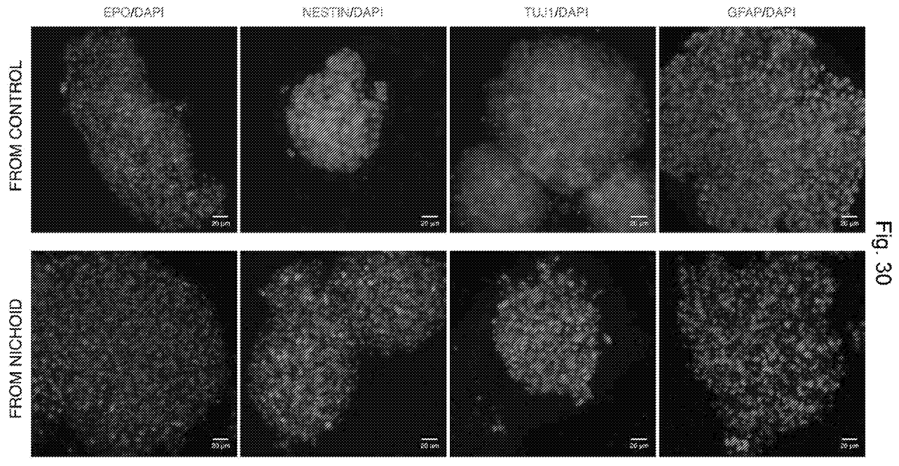

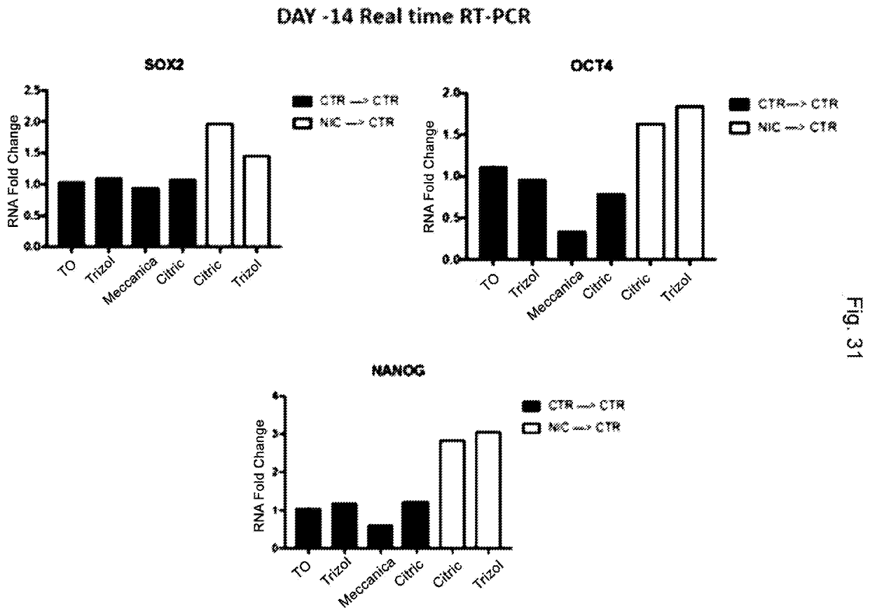

The images of the cells previously cultured within the nichoid vs control were taken on day 1 (FIG. 11), day 3 (FIG. 12) and day 7 (FIG. 13) when they were detached, counted and prepared for both an immunofluorescence dosage and real-time RT-PCR analysis. On day 7, the cells were mechanically dissociated and counted. The plots shown in FIGS. 14, 27 and 28 show that Er-NPCs in the nichoid had a greater proliferation than those maintained under normal fluctuating culture. In fact, they doubled the number of living cells grown under normal conditions, as if they had some sort of memory of their past environment. The thus formed neurospheres were counted and divided based on their size (FIG. 29).

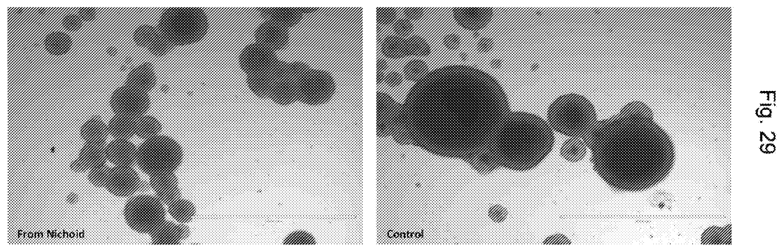

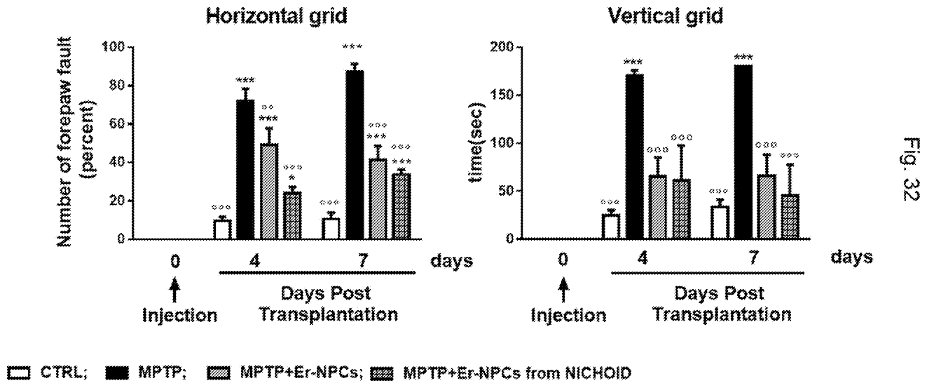

In order to confirm the maintenance of the increased proliferative characteristics, the living cells were re-plated (5×10⁴ cells/cm²) for further 7 days of culture (FIGS. 27, 28, 29 and 30) under the same standard fluctuating conditions but in a larger area using a 24 well plate. Moreover, in the second generation, Er-NPCs grown in the nichoid maintain the feature of growing faster than under standard floating conditions (FIG. 14).

### Example 13

Evaluation of the Marker Expression by Immunofluorescence Assay

An important feature of Er-NPCs is the expression of erythropoietin (EPO) (Marfia et al., 2011). With an immunofluorescence analysis, the expression of markers such as EPO, NESTIN (a classic marker of neural stem cells), TUJ1 (a neuronal marker, Baldassarro et al., 2013) and GFAP (a neural marker) was studied in Er-NPCs cultured for 7 days in the nichoid and then for further 7 days under standard floating conditions. As a control, the comparison with Er-NPCs grown for 14 days under standard floating conditions was carried out (FIG. 31).

### Example 14

### Expression of Staminality Factors (Sox2, Oct4, Nanog) by Real-Time PCR, in Neural Precursors after Seven Days Following the Realtime PCR Under Fluctuating Conditions, Post-Cultivation in the Nichoid

An mRNA analysis was performed on neural precursors cultured for 7 days under standard conditions or in the nichoid and then plated for further 7 days under standard fluctuating conditions (in NSC medium with bFGG and EGF and without serum). The expression of SOX2, OCT4, and NANOG was studied by real-time RT-PCR (FIG. 32).

### Example 15

### Characterization of hADSCs Grown within the Nichoid

This type of cell has never been studied with respect to the nichoid, and therefore it was necessary to evaluate what was the adequate number of cells to be inserted into the substrate and an effective manner to detach them. After that, it was interesting to see what the proliferative power of these cells was within the nichoid, evaluating their viability through an MTT test.

### Proliferation Curve

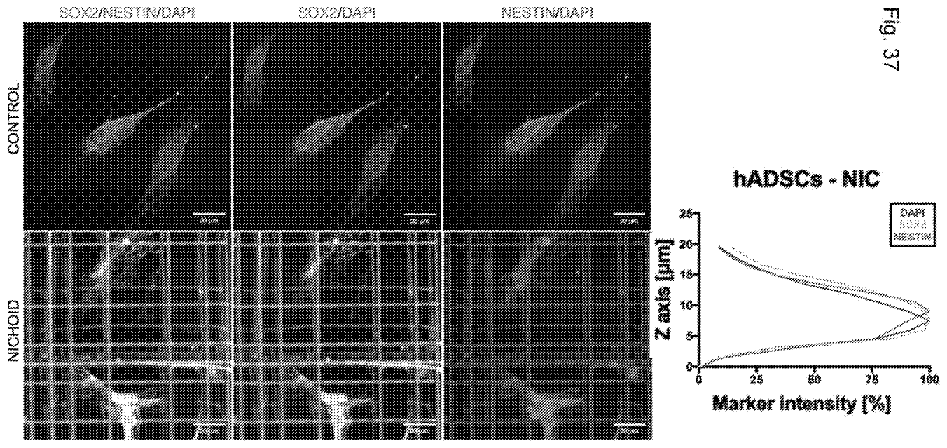

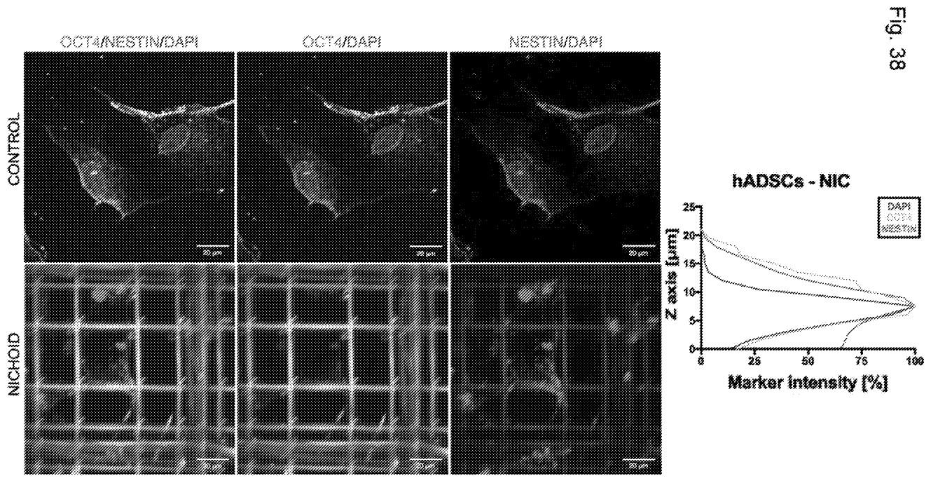

In order to evaluate the proliferative potential of hADSCs grown inside nichoid, 1.5×10³ and 3.5×10³ cells/cm² (FIG. 35) were plated both in the nichoid and under standard control conditions (plastic) for 7 and 14 days, whereafter the cells were detached and counted (FIG. 34). The results showed an increased number of hADSCs when cultured within the nichoid with respect to the control and after 7 and 14 days. This indicates a greater proliferative potential when hADSCs are plated within the nichoid.

### Example 16

Expression of GFAP, VIMENTIN, β-ACTIN, SOX2, NANOG, OCT4, NESTIN

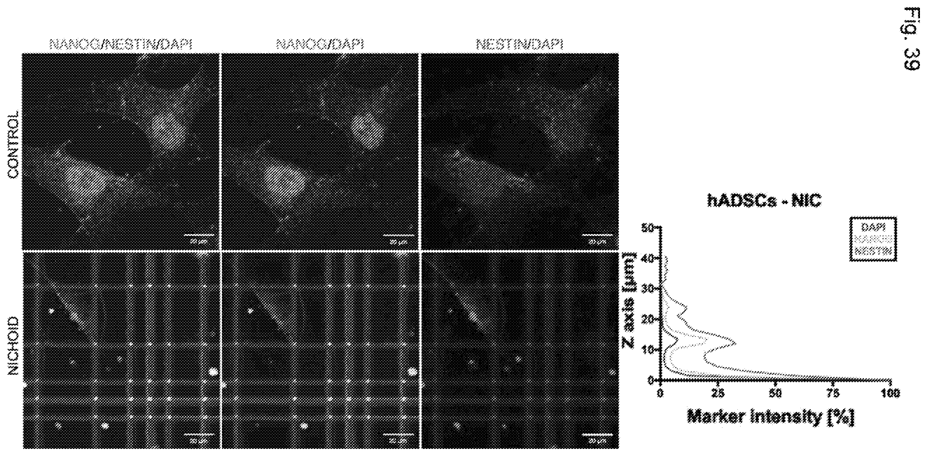

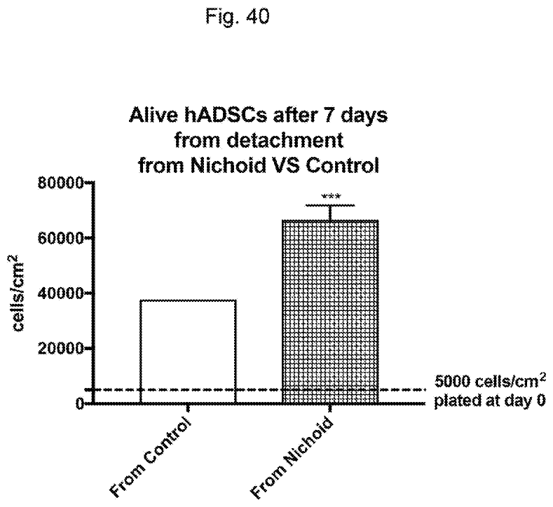

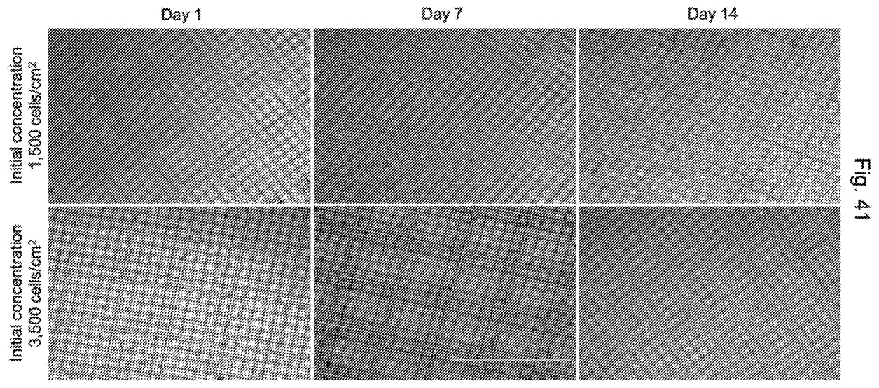

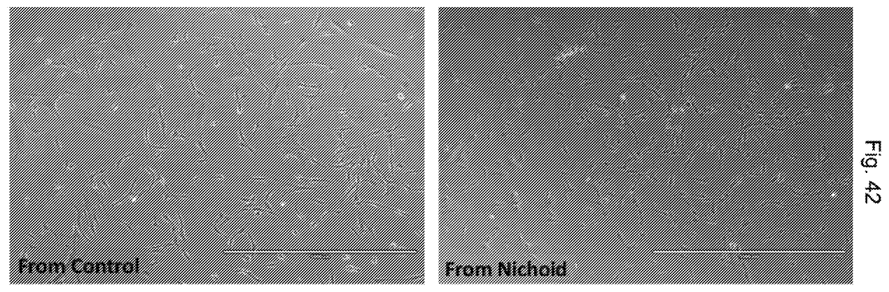

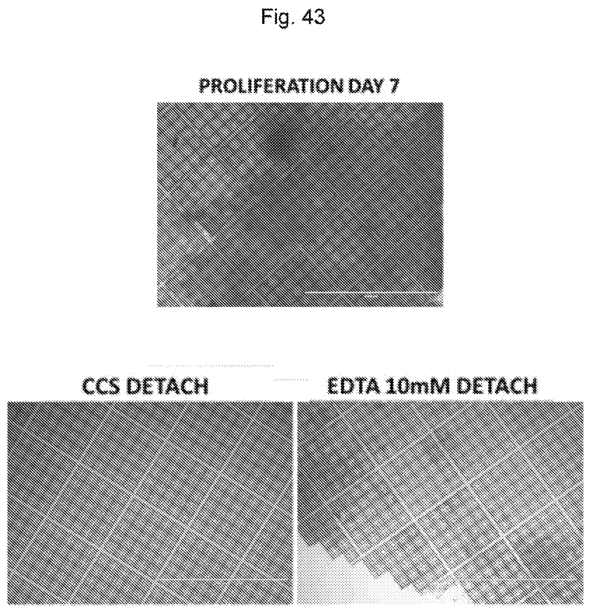

For further characterizing hADSCs grown within the nichoid, the cell marker expression was evaluated by immunofluorescence assays. The markers used were: GFAP, an intermediate-stranded protein expressed by several types of central nervous system cells, and co-expressed with Vimentin, an intermediate-stranded protein which is the main cytoskeletal component of mesenchymal cells (FIG. 36). In the plot, we can see that the co-expression of GFAP and Vimentin is particularly found in the outermost part of the cells, highlighting a wide anchoring effect of the cells on the nichoid structure. GFAP is also co-expressed with β-ACTIN, one of the two non-muscle cytoskeletal actins also involved in cell motility, structure and integrity (FIG. 37). In this case, the expression of the two markers in the nichoid is again present in the outer part of the cells, but with a more planar aspect due to the adhesion to the bottom of the structure as shown in the plot. In order to confirm the ability of the nichoid to maintain the cell staminality, the presence of SOX2, NANOG, OCT4 was studied, which are essential transcription factors for maintaining the self-renewal, or pluripotency, of undifferentiated stem cells co-stained with NESTIN, an intermediate-stranded protein mainly expressed in nerve cells where it is implicated in the radial growth of the axon. These staminality markers were coexpressively evaluated in hADSCs grown for 7 days within the nichoid and under standard conditions. We can see that SOX2 and OCT4 are strongly co-expressed with NESTIN in the nichoid (FIG. 38 and FIG. 39), while NANOG has a different cell distribution in the nichoid with respect to the control (FIG. 40). The plots below demonstrate that the expression of SOX2 and OCT4 is similar to the expression of NESTIN which follows the same intensity trend. Although the expression of NANOG is poorly present in hADSCs grown within the nichoid, it spreads into the closest part to the nucleus unlike NESTIN which is located in the outermost part.

### Example 17

### Comparison of hADSCs Grown within the Nichoid, Detached and Plated Again of with Respect to the Control

The purpose of this trial was to verify the influence of the nichoid on maintaining the proliferation ability of hADSCs grown thereon for one week, detached, and then re-plated under standard conditions for further 7 days. 5×10³ cells/cm², from the nichoid or standard control conditions were plated in a 6 well plate. After 7 days (FIG. 35), they were counted (FIG. 41) by following the usual detachment protocol (Trypsin-0.05% EDTA for 10 minutes). The results showed that the cells plated on plastic from the nichoid maintain their increased proliferative ability (there is indeed a greater number of cells after 7 days).

### Example 18

### The Analysis of RNA Sequencing Reveals Multiple Pathways Involved in Pluripotency

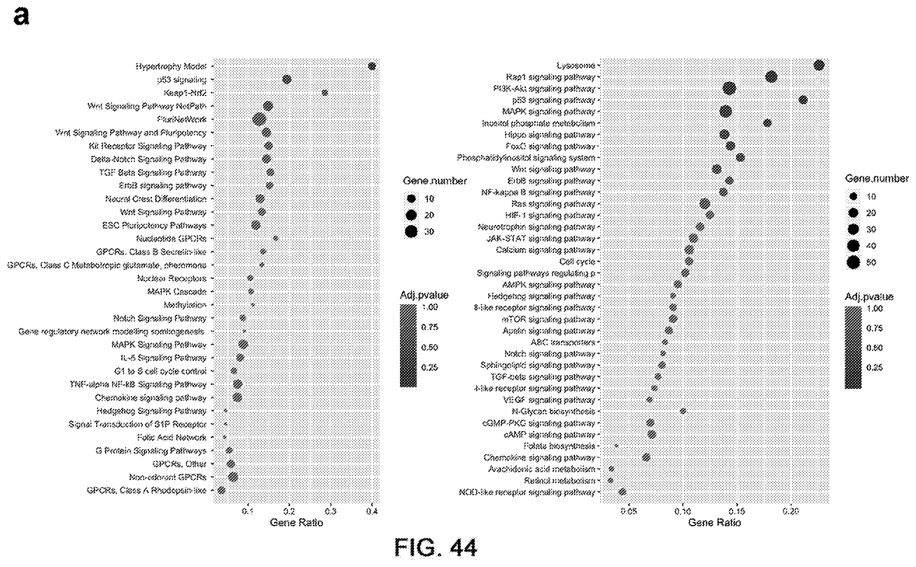

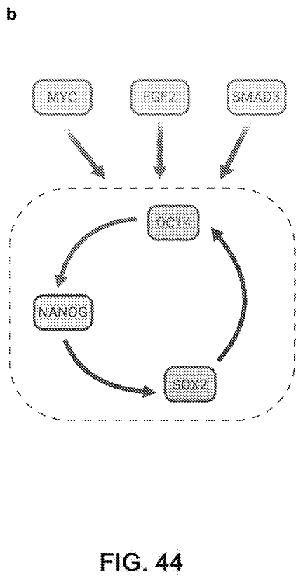

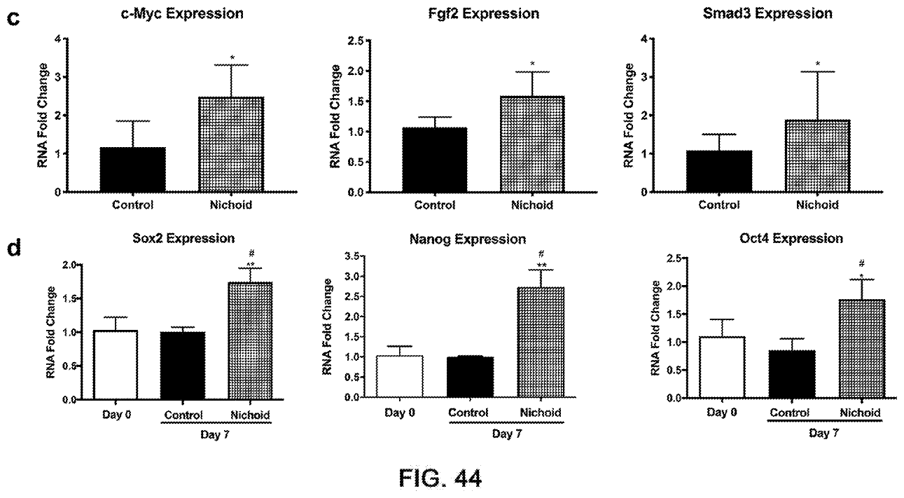

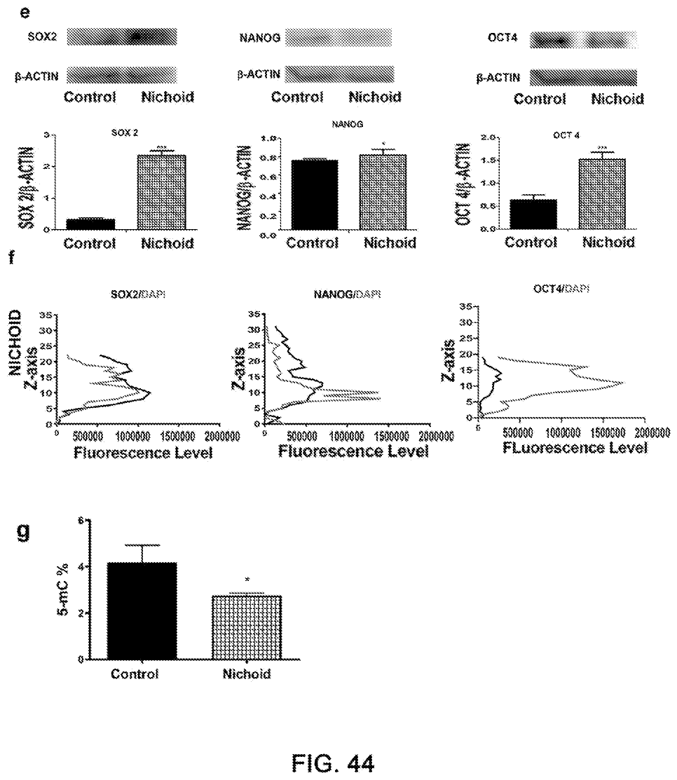

In order to study the effects of the expansion inside the 3D niche on NPCs, a transcriptomic analysis was performed. In particular, NPCs grown within the nichoid for 7 days compared to standard floating conditions (neurospheres) were subjected to RNA sequencing. Among the significantly deregulated pathways, evaluated with the Kyoto Encyclopedia of Genes and Genomes (KEGG) and WikiPathways analyses, it was interesting to see that a large number was correlated with both pluripotency and cell proliferation (FIG. 44A). In particular, out of 277 pathways obtained after KEGG analysis 38 were correlated to pluripotency. This was also confirmed by WikiPathways, where 33 out of 149 paths obtained were correlated to pluripotency (FIG. 44A). This suggests that the nichoid could increase the pluripotency abilities of NPCs by upregulating key genetic regulators of this process. Among these, the presence of c-Myc, Smad3 and Fgf2, key players in the pluripotency pathways found upregulated in NPCs cultured within the nichoid, was observed (FIGS. 44B and 44C). In fact, all these genes converge towards the activation of a transcriptional core network involving SOX2, NANOG and OCT4 and also found upregulated in NPCs expanded within the nichoid (FIG. 44D). These three transcription factors and their downstream target genes promote the self-renewal and pluripotency in a coordinated manner. The expression of SOX2, NANOG and OCT4 was also studied through Western blot and immunofluorescence analysis, confirming that their protein expression has significantly increased (FIG. 44E). The distribution of the three markers (FIG. 44F) shows that NPCs, which are positive for SOX2, NANOG and OCT4 staining are mainly distributed in the lower and central part of the nichoid, suggesting a similarity with the biological niche.

The potential for gene expression in renewal and differentiation in SCs could be regulated by epigenetic processes, of which DNA methylation is the most characterized. In order to investigate the chromatin status of expanded NPCs within the 3D niche, compared to standard floating conditions, the overall levels of DNA methylation were evaluated, which decreased in cells grown within the nichoid with respect to the controls (FIG. 44G). This is a specific feature of multipotent SCs and represents a further evidence of the relevance of the nichoid in increasing the pluri/multipotency ability without exogenous factors.

### Example 19

### Application to Mesenchymal Stem Cells

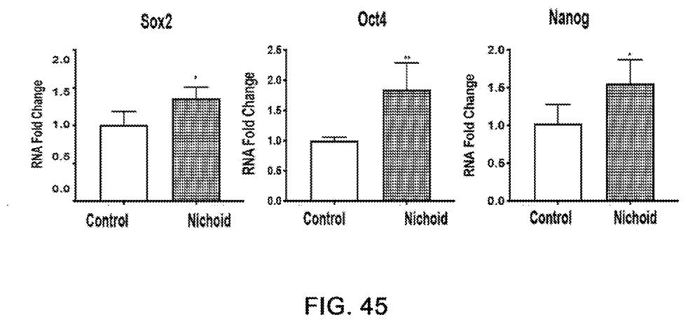

In order to check that the results obtained on neural precursors can also be applied for human mesenchymal stem cells deriving from adipose tissue, three different staminality markers, such as Sox2, Oct4 and Nanog, were analyzed by Real Time PCR. In mesenchymal cells expanded in the nichoid for 7 days, these are significantly more expressed than in control cells expanded in a two-dimensional environment (FIG. 45).

### Example 20

### The Nichoid Increases the In Vivo Therapeutic Efficacy of NPCs

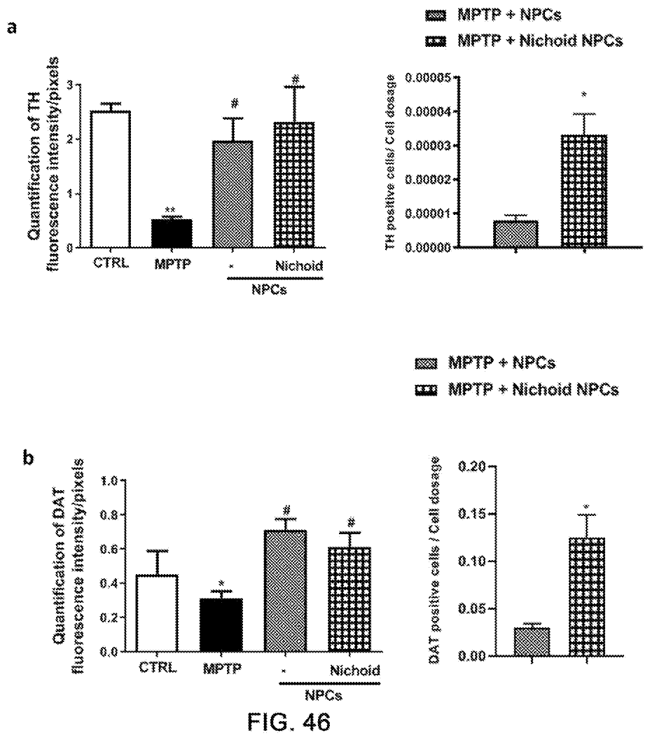

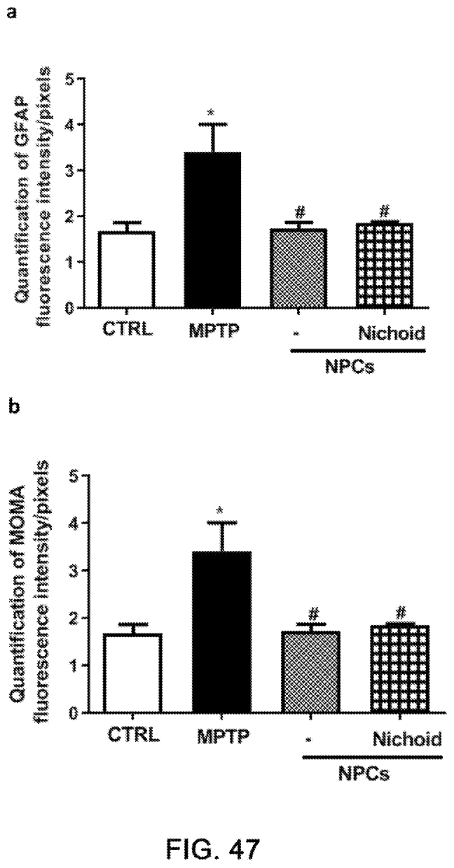

Treatment with MPTP, in the brain (striatum) of the parkinsonian animal, leads to a loss of positivity of the fibers expressing the tyrosine hydroxylase (TH) marker, contrasted by the treatment with NPCs grown both under control conditions and within the nichoid (FIG. 46A). It is possible to notice a greater efficacy for cells grown in the nichoid, considering the relationship with the number of transplanted cells. The same effect is noted for the recovery of positivity for the dopaminergic transporter DAT (FIG. 46B). FIG. 47 shows instead an increase in expressions of the neuroinflammation markers GFAP and MOMA in the striatum sections of parkinsonian mice (MPTP), contrasted by the infusion with NPCs grown under standard conditions and within the nichoid.

What is claimed is:

1. A method for inducing pluripotency in cells by using a nichoid-type substrate.

2. The method of claim 1, wherein said cells are stem cells.

3. The method of claim 2, wherein said stem cells are adult, embryonic, cordonal, placental, or fetal stem cells.

4. The method of claim 3, wherein said adult stem cells are neural progenitors or mesenchymal cells.

5. The method of claim 1, comprising the steps of:
a) plating said cells on said nichoid-type substrate; and
b) allowing cultured cells to proliferate for a certain period of time.

6. The method of claim 5, wherein in step b), proliferation is carried out for a period of time between about 1 day and 10 days.

7. The method of claim 5, further comprising the step of:
c) detaching said cells from the nichoid-type substrate.

8. The method of claim 7, wherein said cells are detached from the nichoid-type substrate by a sodium citrate solution.

9. The method of claim 7, wherein once the cells have been detached, in a step d) they are cultured in suspension or under adhesion.

10. The method of claim 1, wherein said cells are Erythropoietin-releasing Neural Precursor Cells (Er-NPCs).

11. A method for therapeutic treatment in a subject, said method comprising administering to said subject stem cells obtained according to the method of claim 1.

12. (canceled)

13. The method of claim 11, wherein said stem cells are administered by intracerebral, intraspinal or intravenous transplantation.

14. A method of differentiating stem cells comprising the steps of:
i) plating said cells on a nichoid-type substrate in the presence of a culture medium which does not comprise serum,
ii) replacing the culture medium with a medium which comprises serum, and
iii) harvesting the differentiated cells,
wherein step i) is carried out in the absence of a cell adhesion-promoting substrate.

15. The method of claim 14, wherein said cells are neural progenitor cells.

16. The method of claim 15, wherein said cells are Erythropoietin-releasing Neural Precursor Cells (Er-NPCs).

17. The method of claim 14, wherein in step ii) said medium comprises about 2% serum.

18. (canceled)

19. (canceled)

20. A method for treating neurodegenerative diseases in a subject in need thereof, said method comprising administering to said subject neuronal cells obtained according to the method of claim 14.

21. The method of claim 20, wherein said neuronal cells are administered by intracerebral, intraspinal or intravenous transplantation.
